(19) United States
(12) Patent Application Publication
(10) Pub. No.: US 2010/0034791 A1
(43) Pub. Date: Feb. 11, 2010

(54) Engineered Lung Tissue Construction for High Throughput Toxicity Screening and Drug Discovery

(75) Inventors: Peter Lelkes, Cherry Hill, NJ (US); Christine M. Pinck, Glastonbury, CT (US); Mark J. Mondrinos, Lansdowne, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: DREXEL UNIVERSITY

(21) Appl. No.: 12/526,908

(22) PCT Filed: Feb. 14, 2008
(86) PCT No.: PCT/US08/01935

§ 371 Date: Aug. 12, 2009

Related U.S. Application Data
(60) Provisional application No. 60/889,779, filed on Feb. 14, 2007.

(51) Int. Cl. A61K 35/12; C12N 11/02; C12Q 1/02

(52) U.S. Cl. 424/93.21; 435/177; 435/29; 424/93.7

(57) ABSTRACT

The present invention relates to compositions comprising fetal pulmonary cells and biocompatible materials. The present invention also provides an engineered three dimensional lung tissue exhibiting characteristics of a natural lung tissue. The engineered tissue is useful for the study of lung developmental biology and pathology as well as drug discovery.

### BACKGROUND OF THE INVENTION

Pulmonary hypoplasia is found in as many as 15-20% of all neonatal autopsies. The pathology of pulmonary hypoplasia and resultant pediatric pulmonary conditions, such as bronchopulmonary dysplasia, are hallmarked by aberrant vascular and epithelial development. In addition, adult pulmonary diseases, such as emphysema, are characterized by destruction of epithelial and vascular tissues, culminating in respiratory distress.

Congenital diaphragmatic hernia (CDH) occurs in about 1 in 3000 human live births. Although it is associated with several genetic defects, its exact etiology is not known. Newborns with CDH have a 40-50% mortality, which is primarily caused by the associated pulmonary hypoplasia. The hypoplastic lungs are not capable of providing adequate gas exchange for oxygenation, and persistent pulmonary hypertension leads to refractory hypoxia (right to left shunting). Unlike other causes of neonatal respiratory failure, infants with CDH are often unresponsive to the modern therapeutic armamentarium, because it does not solve the basic problem of lung hypoplasia (Thbaud et al., 1998 Biol. Neonate 74:323-336).

Since the first description of Nitrofen-induced diaphragmatic hernias in rodents by Iritani in 1984, the murine nitrofen-induced model of CDH has been extensively studied, and by now is widely accepted as a well-established model that has many phenotypic similarities to the human condition (Iritani, 1984 Anat Embryol 169:133-9; Greer et al., 2000 Pediatric Pulmonol 29:394-9; Kluth et al., 1990 J Pediatr Surg 25:850-4). Using this model in mice, it has been shown that Nitrofen causes primary pulmonary hypoplasia, which is worsened by the presence of a hernia (Coleman et al., 1998 Am J Physiol 274:636-646). In rats, Nitrofen has also recently been shown to reduce branching morphogenesis before diaphragmatic closure, both in vitro and in vivo (Keijzer et al., 2000 Am J Path 156:1299-1306). Since Nitrofen-exposed embryonic lungs are clearly hypoplastic prior to the appearance of an actual diaphragmatic defect, an evaluation of candidate factors known to be required for early lung development was initiated (Warburton et al., 2000 Mech Dev. 92:55-81).

During mouse lung morphogenesis, the distal mesenchyme has long been known to regulate the growth and branching of the adjacent endoderm through the secretion of soluble factors (Warburton et al, 2000 Mech Dev. 92:55-81). Bellusci et al (1997 Development 124:4867-78) reported that FGF 10 is a mesenchyme-derived factor that plays a critical role in patterning the early branching events in lung development. Fgf10 null mutant mice and transgenic mice expressing dominant negative forms of the FGF10 receptor, Fgfr2-IIIb, have a dramatic inhibition of bronchial branching (Min et al., 1998 Genes Dev 12:3156-61; Peters et al., 1994 EMBO J 13:3296-3301). Fgf10 is expressed in a temporospatially specific pattern in the peripheral embryonic lung mesenchyme near the positions where primary, secondary and tertiary bronchi bud (Bellusci et al., 1997 Development 124:4867-78). The buds grow towards these areas of Fgf10 expression. Thus Fgf10 appears to stimulate and direct early bronchial branching. FGF-pathway signaling is modified at each stage of branching by genetic feedback controls. Sonic hedgehog (Shh), which is strongly expressed in the distal epithelium, may function as a negative signal for Fgf10 (Bellusci et al., 1997 Development 124:53-63; Grindley et al., 1997 Dev Biol 188:337-348). Shh inhibits Fgf10 expression in the mesenchyme near growing tips, where the initial Fgf10 expression domain splits laterally into two domains. Two new buds then sprout, each targeting one of the lateral subdomains of Fgf10 expression. Mice in whom Shh has been inactivated also have profound impairments of lung branching (Pepicelli et al., 1998 Curr Biol 8:1083-1086). Other key antagonists of the FGF-pathway include members of the Sprouty gene family. Murine Sprouty 2 (mSpry2) is an inducible negative regulator of FGF receptor tyrosine kinase signaling that is expressed in the distal epithelium of the embryonic mouse lung, adjacent to the mesenchymal loci of Fgf10 expression, at embryonic stages when lung epithelial buds are highly responsive to FGF10. Abrogation of mSpry2 expression in lung organ cultures with antisense oligonucleotides increases branching morphogenesis and surfactant gene expression (Tefft et al., 1999 Curr Biol 9:219-22).

Alveolar epithelial type 2 cells (AEC2) have been designated the primary progenitor cell of the alveolar epithelium (Ten Have-Opbroek, 1979 Dev. Biol. 69:408-423). In the embryo, AEC2 arise from multipotent stem cells which line the primitive respiratory tract. These primitive, proliferative embryonic epithelial precursors co-express several markers, including SP-A, SP-C, CC.10 and cGRP, which are subsequently expressed in separate, differentiated lineages in the mature fetus and in the adult, including AEC2, Clara cells and pulmonary neuroendocrine cells (Wuenschell et al., 1996 J. Histochem. Cytochem. 44:113-123). At late gestation, the AEC lineage becomes restricted, such that only AEC type 1 and type 2 cells are produced (Mason et al., 1997 Am. J. Respir. Cell Mol. Biol. 16:355-363). Type 2 cells manufacture surfactant and can differentiate, as required, into AEC1 (Ten Have-Opbroek, et al., 1991 Anat. Rec. 229:339-354). AEC1 are terminally differentiated, incapable of dividing, and perform the necessary lung function of gas exchange. However, the ability to divide must be retained by a sub-population within the lung alveolar epithelium throughout the life span of any animal, in order to replace damaged cells (Adamson and Bowden, 1974 Lab Invest. 30:35-42; Evans, et al. 1975 Exp. Mol. Pathol. 22:142-150). This stem or progenitor cell function has been FPCribed to AEC2.

Worldwide, much investigation has been done on lung cells and diseases which affect lung cells, for instance emphysema and lung cancer. Until now, however, there is no efficient treatment of emphysema and lung cancer. In the case of emphysema, patients suffer from shortness of breath, in first instance only on exertion, later on also at rest. This symptom may be accompanied by coughing, often with mucus expectorated. In later stages of the disease, heart failure occurs due to low oxygen levels in the blood circulation, often presenting as swollen ankles and liver enlargement. Pulmonary symptoms can be reduced by bronchodilator therapy and by use of courses of oral steroids. End-stage disease is treated with supplementation of oxygen by nasal canula. There is no treatment for the underlying cause of the disease. Consequently, most attention is being paid to decrease or even stop the process of dying of lung cells. Although some result has been obtained by the use of inhaled steroids, the lung damage continues which causes a progressive decrease in function (Pauwels et al., 1999; Burge, 2000). The problem is that even if the lung diseases can be counteracted, the lungs are already damaged by the disease.

Conventional animal tests employed to evaluate new therapeutic agents or identify suspect disease associated targets are expensive, time consuming, require skilled animal-trained staff and utilize large numbers of animals. To date in vitro alternatives have relied on the use of conventional cell culture systems which are limited in that they do not allow the three-dimensional interactions that occur between lung cells and with their surrounding tissue. This is a serious disadvantage as such interactions are well documented as having a significant influence on the growth and invasion profiles of lung disease.

Accordingly, there is a great need for more sensitive and accurate methods for predicting whether a person is likely to develop a lung disease or disorder, for diagnosing a lung disease or disorder, for monitoring the progression of the disease or disorder, and the like. There is also a need for better treatment of lung disease or disorder such as lung cancer, emphysema, pneumonia, lung infection, pulmonary fibrosis, cystic fibrosis, and asthma.

### BRIEF SUMMARY OF THE INVENTION

The invention provides a composition comprising a three dimensional scaffold and a population of fetal pulmonary cells (FPCs), wherein the composition is capable of supporting and maintaining the differentiation state of an alveolar epithelial cell.

In one embodiment, the population of FPCs comprises epithelial, mesenchymal, and endothelial cells. In another embodiment, the cells are genetically modified.

In one embodiment, the composition further comprises fibroblast growth factor (FGF), wherein the FGF is selected from the group consisting of FGF2, FGF7, FGF10, and any combination thereof.

In one embodiment, the scaffold comprises a biocompatiable material selected from the group consisting of fibronectin, laminin, collagen, glycoprotein, thrombospondin, elastin, fibrillin, mucopolysaccharide, glycolipid, heparin sulfate, chondroitin sulfate, keratin sulfate, glycosaminoglycan, hyaluronic acid, proteoglycan, vitronectin, poly-D-lysine, polysaccharide, and any combination thereof.

The invention also provides an engineered three dimensional construct, wherein construct is capable of supporting and maintaining the differentiation state of an alveolar epithelial cell.

In one embodiment, the construction comprises a population of FPCs, wherein the population of FPCs comprises epithelial, mesenchymal, and endothelial cells. In another embodiment, FPCs are genetically modified.

In one embodiment, the construct comprises FGF, wherein the FGF is selected from the group consisting of FGF2, FGF7, FGF10, and any combination thereof.

In one embodiment, the construct comprises cells that exhibit gene expression associated with induction of branching morphogenesis. In another embodiment, the gene is selected from the group consisting of surfactant protein C (SpC), SpB, FGF10, fibroblast growth factor receptor 2 (FGFr2), vascular endothelial growth factor A (VEGF), and any combination thereof.

In one embodiment, the construct comprises a characteristic of a lung tissue, wherein the characteristic is selected from the group consisting of branching morphogenesis, distal lung epithelial cytodifferentiation, epithelial budding, epithelial growth, vascular development, and any combination thereof.

In one embodiment, the construct is in a mammal.

In one embodiment, the construct comprises a biocompatiable material selected from the group consisting of fibronectin, laminin, collagen, glycoprotein, thrombospondin, elastin, fibrillin, mucopolysaccharide, glycolipid, heparin sulfate, chondroitin sulfate, keratin sulfate, glycosaminoglycan, hyaluronic acid, proteoglycan, vitronectin, poly-D-lysine, polysaccharide, and any combination thereof.

The invention provides a method of making an engineered three dimensional construct capable of supporting and maintaining the differentiation state of an alveolar epithelial cell. The method comprises seeding a scaffold with a population of FPCs to produce a seeded scaffold.

In one embodiment, the population of FPCs comprises epithelial, mesenchymal, and endothelial cells.

In one embodiment the FPCs have been cultured in the presence of FGF for a period of time prior to seeding, wherein the FGF is selected from the group consisting of FGF2, FGF7, FGF10, and any combination thereof.

In one embodiment, the FPCs are seeded in the presence of FGF, wherein the FGF is selected from the group consisting of FGF2, FGF7, FGF10, and any combination thereof.

In one embodiment, the scaffold comprises a biocompatiable material selected from the group consisting of fibronectin, laminin, collagen, glycoprotein, thrombospondin, elastin, fibrillin, mucopolysaccharide, glycolipid, heparin sulfate, chondroitin sulfate, keratin sulfate, glycosaminoglycan, hyaluronic acid, proteoglycan, vitronectin, poly-D-lysine, polysaccharide, and any combination thereof.

The invention provides an in vitro method for screening a test agent for the ability of the test agent to modulate the health of a lung tissue. The method comprises contacting a test agent to an engineered three dimensional lung tissue model and measuring the effect the test agent has on the model, wherein any alteration to the model is an indication that the test agent is able to modulate the health of a lung tissue.

In one embodiment, the test agent is selected from the group consisting of a chemical agent, a pharmaceutical, a peptide, a nucleic acid, and radiation.

In one embodiment, the test agent is a delivery vehicle for a therapeutic agent.

In one embodiment, the method comprises determining the effect of the test agent on cell number, area, volume, shape, morphology, marker expression or chromosomal fragmentation.

In one embodiment, the method comprises the step of selecting an agent which has a desired effect on the lung tissue model.

The invention provides a method of alleviating or treating a lung defect in a mammal. The method comprises administering to a mammal a therapeutically effective amount of a composition comprising a three dimensional construct capable of supporting and maintaining the differentiation state of an alveolar epithelial cell, thereby alleviating or treating the lung defect in the mammal.

In one embodiment, the construct comprises a population of FPCs, wherein the population of FPCs comprises epithelial, mesenchymal, and endothelial cells. In another embodiment, the FPCs are genetically modified.

In one embodiment, the construct comprises FGF, wherein the FGF is selected from the group consisting of FGF2, FGF7, FGF10, and any combination thereof.

In one embodiment, the construct comprises cells that exhibit gene expression associated with induction of branching morphogenesis. In another embodiment, the gene is selected from the group consisting of surfactant protein C (SpC), SpB, FGF10, FGFr2, vascular endothelial growth factor A (VEGF), and any combination thereof.

In one embodiment, the construct comprises a characteristic of a lung tissue, wherein the characteristic is selected from the group consisting of branching morphogenesis, distal lung epithelial cytodifferentiation, epithelial budding, epithelial growth, vascular development, and any combination thereof.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A-1G is a series of images showing epithelial growth and morphology as a function of FGF supplementation. FIGS. 1A-1F are representative phase contrast micrographs of AFUs following 7 days of culture in the presence of FG10, FGF7 and FGF2 alone and in combination. FIG. 1G is a chart depicting that quantification of AFU growth in response to FGF supplementation.

FIG. 2, comprising FIGS. 2A-2M, is a series of images depicting epithelial morphogenesis and cytodifferentiation. FIGS. 2A-2L are representative optical sections through AFUs stained for cytokeratin (red) to visualize epithelial cells and counterstained with DAPI (blue) for nuclei. FIG. 2M is a chart depicting the quantification of epithelial cell numbers comprising AFUs, as measured by counting DAPI stained nuclei and mesenchymal cell numbers by counting tropoelastin positive cells in 400× microscopic fields of interstitial spaces as shown in FIG. 2L.

FIG. 3, comprising FIGS. 3A-3F, is a series of images showing identification of endothelial cells in FPC populations.

FIG. 4, comprising FIGS. 4A-4H, is a series of images demonstrating FGF dependence of vascular morphogenesis following 7 days of in vitro culture, as assessed by fluorescent confocal microscopy. FIGS. 4A-4G are images of endothelial cells within constructs across FGF supplementation conditions. FIG. 4H is a chart depicting the quantitative image analysis of isolectinB4 staining of endothelial network formation across FGF supplementation conditions.

FIG. 5, comprising FIGS. 5A-5F, is a series of images depicting the visualization of epithelial-endothelial interfacing by fluorescent confocal microscopy of whole mount stained constructs across FGF supplementation conditions.

FIG. 6, comprising FIGS. 6A-6D, is a series of images depicting the viability staining and gene expression analysis of collagen gel constructs across FGF supplementation conditions.

FIG. 7, comprising FIGS. 7A-7H, is a series of images demonstrating the detection of FGF receptors, FGFR1 and FGFR2 in the cultured FPC.

FIG. 8, comprising FIGS. 8A-8H, is a series of images depicting the histology of in vivo engineered pulmonary tissue constructs.

FIG. 9, comprising FIGS. 9A-9D, is a series of images depicting immunohistochemical staining of engrafted FPCs.

FIG. 10, comprising FIGS. 10A-10E, is a series of images depicting the visualization and quantification of patent vasculature within Matrigel plugs.

FIG. 11, comprising FIGS. 11A-11B, is a series of images demonstrating that FPC-derived ECs contribute to the formation of TITC-dextran-perfused vessels within MG+FPCs+FGF2 construct generated over 7 days in vivo.

### DETAILED DESCRIPTION OF THE INVENTION

The present invention is partly based on the discovery that a three dimensional lung tissue can be generated to exhibit characteristics of a natural lung tissue. For example, the invention provides a method of maintaining the differentiation state of alveolar epithelial cells for extended period of time in vitro and the induction of genes related to morphogenetic processes for lung tissue. A non-limiting epithelial gene related to the branching morphogenesis is fibroblast growth factor receptor 2 (FGFr2).

The in vitro three dimensional model of lung tissue of the invention is useful for investigating lung developmental biology. In addition, the model is useful for among other things, drug discovery, toxicity testing, disease pathology, and the like.

The invention is also related to the discovery that lung tissue can be generated in vivo. The in vivo model recapitulates the formation of structures reminiscent of alveolar forming units comprised of ductal epithelium tightly interfaced with the host circulation. Accordingly, the invention provides methods and compositions for the generation of vascularized pulmonary tissues as a form of regenerative medicine.

The invention also provides a method of alleviating or treating a lung defect in a mammal, preferably a human. The method comprises administering to the mammal in need thereof a therapeutically effective amount of a composition comprising a three dimensional construct of the invention, thereby alleviating or treating the lung defect in the mammal.

Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

Standard techniques are used for nucleic acid and peptide synthesis. The techniques and procedures are generally performed according to conventional methods in the art and various general references (e.g., Sambrook and Russell, 2001, Molecular Cloning, A Laboratory Approach, Cold Spring Harbor Laboratory Press, Cold Spring Harbor, N.Y., and Ausubel et al., 2002, Current Protocols in Molecular Biology, John Wiley & Sons, New York, N.Y.), which are provided throughout this document.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent based on the context in which it is used.

The terms “precursor cell,” “progenitor cell,” and “stem cell” are used interchangeably in the art and as used herein refer either to a pluripotent or lineage-uncommitted progenitor cell, which is potentially capable of an unlimited number of mitotic divisions to either renew itself or to produce progeny cells which will differentiate into the desired cell type. In contrast to pluripotent stem cells, lineage-committed progenitor cells are generally considered to be incapable of giving rise to numerous cell types that phenotypically differ from each other. Instead, progenitor cells give rise to one or possibly two lineage-committed cell types.

The term “dedifferentiation”, as used herein, refers to the return of a cell to a less specialized state. After dedifferentiation, such a cell will have the capacity to differentiate into more or different cell types than was possible prior to re-programming. The process of reverse differentiation (i.e., de-differentiation) is likely more complicated than differentiation and requires “re-programming” the cell to become more primitive.

As used herein, “scaffold” refers to a structure, comprising a biocompatible material, that provides a surface suitable for adherence and proliferation of cells. A scaffold may further provide mechanical stability and support. A scaffold may be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal.

As used herein, “autologous” refers to a biological material derived from the same individual into whom the material will later be re-introduced.

As used herein, “allogeneic” refers to a biological material derived from a genetically different individual of the same species as the individual into whom the material will be introduced.

As used herein, a “graft” refers to a cell, tissue or organ that is implanted into an individual, typically to replace, correct or otherwise overcome a defect. A graft may further comprise a scaffold. The tissue or organ may consist of cells that originate from the same individual; this graft is referred to herein by the following interchangeable terms: “autograft”, “autologous transplant”, “autologous implant” and “autologous graft”. A graft comprising cells from a genetically different individual of the same species is referred to herein by the following interchangeable terms: “allograft”, “allogeneic transplant”, “allogeneic implant” and “allogeneic graft”. A graft from an individual to his identical twin is referred to herein as an “isograft”, a “syngeneic transplant”, a “syngeneic implant” or a “syngeneic graft”. A “xenograft”, “xenogeneic transplant” or “xenogeneic implant” refers to a graft from one individual to another of a different species.

As used herein, the terms “tissue grafting” and “tissue reconstructing” both refer to implanting a graft into an individual to treat or alleviate a tissue defect, such as a lung defect or a soft tissue defect.

As used herein, to “alleviate” a disease, defect, disorder or condition means reducing the severity of one or more symptoms of the disease, defect, disorder or condition.

As used herein, to “treat” means reducing the frequency with which symptoms of a disease, defect, disorder, or adverse condition, and the like, are experienced by a patient.

As used herein, a “therapeutically effective amount” is the amount of a composition of the invention sufficient to provide a beneficial effect to the individual to whom the composition is administered.

As used herein, the term “growth medium” is meant to refer to a culture medium that promotes growth of cells. A growth medium will generally contain animal serum. In some instances, the growth medium may not contain animal serum.

“Differentiation medium” is used herein to refer to a cell growth medium comprising an additive or a lack of an additive such that a stem cell, fetal pulmonary cell or other such progenitor cell, that is not fully differentiated, develops into a cell with some or all of the characteristics of a differentiated cell when incubated in the medium.

As used herein, the term “growth factor product” refers to a protein, peptide, mitogen, or other molecule having a growth, proliferative, differentiative, or trophic effect on a cell. Growth factors include, but are not limited to, fibroblast growth factor (FGF), basic fibroblast growth factor (bFGF), acidic fibroblast growth factor (aFGF), epidermal growth factor (EGF), insulin-like growth factor-I (IGF-T), insulin-like growth factor-II (IGF-II), platelet-derived growth factor (PDGF), vascular endothelial cell growth factor (VEGF), activin-A, bone morphogenic proteins (BMPs), insulin, growth hormone, erythropoietin, thrombopoietin, interleukin 3 (IL-3), interleukin 6 (IL-6), interleukin 7 (IL-7), macrophage colony stimulating factor, c-kit ligand/stem cell factor, osteoprotegerin ligand, insulin, nerve growth factor, ciliary neurotrophic factor, cytokines, chemokines, morphogens, neutralizing antibodies, other proteins, and small molecules. Preferably, the FGF is selected from the group selected from FGF2, FGF7, FGF10, and any combination thereof.

An “isolated cell” refers to a cell which has been separated from other components and/or cells which naturally accompany the isolated cell in a tissue or mammal.

As used herein, a “fetal pulmonary cells” (FPCs) refer to cells isolated from the lung tissue of an embryo. A mixed population of FPCs can include, but is not limited to epithelial, mesenchymal, and endothelial cells.

As used herein, “epithelial cell” means a cell which forms the outer surface of the body and lines organs, cavities and mucosal surfaces.

As used herein, “endothelial cell” means a cell which lines the blood and lymphatic vessels and various other body cavities.

As used herein, a “substantially purified” cell is a cell that is essentially free of other cell types. Thus, a substantially purified cell refers to a cell which has been purified from other cell types with which it is normally associated in its naturally-occurring state.

“Expandability” is used herein to refer to the capacity of a cell to proliferate, for example, to expand in number or, in the case of a population of cells, to undergo population doublings.

The term “lung specific” refers to a nucleic acid molecule or polypeptide that is expressed predominantly in the lung as compared to other tissues in the body. In a preferred embodiment, a “lung specific” nucleic acid molecule or polypeptide is expressed at a level that is 5-fold higher than any other tissue in the body. In a more preferred embodiment, the “lung specific” nucleic acid molecule or polypeptide is expressed at a level that is 10-fold higher than any other tissue in the body, more preferably at least 15-fold, 20-fold, 25-fold, 50-fold or 100-fold higher than any other tissue in the body. Nucleic acid molecule levels may be measured by nucleic acid hybridization, such as Northern blot hybridization, or quantitative PCR. Polypeptide levels may be measured by any method known to accurately measure protein levels, such as Western blot analysis.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine into the cell, and the like.

As used herein, “tissue engineering” refers to the process of generating tissues ex vivo for use in tissue replacement or reconstruction. Tissue engineering is an example of “regenerative medicine,” which encompasses approaches to the repair or replacement of tissues and organs by incorporation of cells, gene or other biological building blocks, along with bioengineered materials and technologies.

As used herein “endogenous” refers to any material from or produced inside an organism, cell or system.

“Exogenous” refers to any material introduced into or produced outside an organism, cell, or system.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally-occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The phrase “under transcriptional control” or “operatively linked” as used herein means that the promoter is in the correct location and orientation in relation to the polynucleotides to control RNA polymerase initiation and expression of the polynucleotides.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a cell substantially only if the cell is a cell of the tissue type corresponding to the promoter.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (i.e., naked or contained in liposomes) and viruses that incorporate the recombinant polynucleotide.

The term “patient” as used herein includes human and veterinary subjects.

Description of the Invention

The present invention provides an engineered three dimensional pulmonary tissue and methods of making the three dimensional pulmonary tissue. Preferably, the pulmonary tissue is a lung tissue. In one embodiment, the engineered pulmonary tissue exhibits branching morphogenesis exemplified by natural pulmonary tissue. Thus, the invention provides an in vitro model that mimics natural pulmonary tissue. The in vitro three dimensional pulmonary tissue model is useful for among other things, drug discovery, toxicity testing, disease pathology, and the like.

The engineered three dimensional pulmonary tissue comprises fetal pulmonary cells (FPCs). In some instances, a mixed population of FPCs are used, wherein the population of FPCs include, but are not limited to epithelial cells, mesenchymal cells, and endothelial cells.

The invention also includes generation of pulmonary tissue in vivo. Preferably, vascularized pulmonary tissue is generated in vivo. In one aspect, the fetal pulmonary cells are administered to a mammal to facilitate in vivo pulmonary tissue formation.

In the present invention, it is demonstrated that biocompatible scaffolds can be seeded with FPCs and the resultant composition can be used as a vascularized three dimensional pulmonary tissue model for preclinical in vitro pharmacological, physiological, and scientific testing. In addition, the biocompatible scaffolds can be seeded with FPCs and the resultant composition can be used for tissue reconstruction in vivo.

The FPCs may be induced to differentiate prior to implantation for tissue reconstruction (i.e. ex vivo) or may be induced to differentiate after implantation (i.e. in vivo). In a preferred embodiment, three dimensional hydrogels can be used to make a biocompatible scaffold which is seeded with FPC. After seeding, the cells on the scaffold are optionally subjected to an expansion medium or to a differentiation medium or cultured in the presence of tissue-specific growth factors. The composition is then implanted into a subject in need thereof. The subject may be a mammal, but is preferably a human and the source of the cells for growth and implantation is any mammal, preferably a human. The implanted composition supports additional cell growth in vivo, thus providing tissue reconstruction. Accordingly, the invention provides the use of engineered three dimensional pulmonary tissue for tissue grafting therapies.

The compositions and methods of the instant invention have myriad useful applications. The compositions may be used in therapeutic methods for alleviating or treating tissue defects in an individual. The compositions may also be used in vitro or in vivo to identify therapeutic compounds and therefore may have therapeutic potential.

### Isolating and Expanding FPCs

The compositions and methods of the instant invention can be practiced using fetal pulmonary cells (FPCs). Preferably, the FPCs are isolated from a mammal, more preferably a primate and more preferably still, a human.

The FPCs useful in the methods of the present invention are isolated using methods discussed herein, for example in the Examples section, or by any method known in the art. FPCs are isolated from the lung of an embryo of a mammal. Following isolation, the FPC are cultured in a culture medium.

Any medium capable of supporting fibroblasts in cell culture may be used as a culture medium. Media formulations that support the growth of fibroblasts include, but are not limited to, Minimum Essential Medium Eagle, ADC-1, LPM (bovine serum albumin-free), F10 (HAM), F12 (HAM), DCCM1, DCCM2, RPMI 1640, BGJ Medium (with and without Fitton-Jackson Modification), Basal Medium Eagle (BME-with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's salt base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with nonessential amino acids), and the like. A preferred medium for culturing FPCs is DMEM, more preferably DMEM/F12.

Additional non-limiting examples of media useful in the methods of the invention may contain fetal serum of bovine or other species at a concentration at least 1% to about 30%, preferably at least about 5% to 15%, most preferably about 10%. Embryonic extract of bovine or other species can be present at a concentration of about 1% to 30%, preferably at least about 5% to 15%, most preferably about 10%.

Typically, the FPC culture medium comprises a base medium, serum and an antibiotic/antimycotic. The preferred base medium is DMEM/F12 (1:1). The preferred serum is fetal bovine serum (FBS) but other sera may be used, including horse serum or human serum. Preferably up to 20% FBS will be added to the above medium in order to support the growth of FPCs. However, a defined medium can be used if the necessary growth factors, cytokines, and hormones in FBS for FPC growth are identified and provided at appropriate concentrations in the growth medium. It is further recognized that additional components may be added to the culture medium. Such components include, but are not limited to, antibiotics, antimycotics, albumin, growth factors, amino acids, and other components known to the art for the culture of cells. Antibiotics which can be added into the medium include, but are not limited to, penicillin and streptomycin. The concentration of penicillin in the culture medium is about 10 to about 200 units per ml. The concentration of streptomycin in the culture medium is about 10 to about 200 μg/ml. However, the invention should in no way be construed to be limited to any one medium for culturing FPCs. Rather, any media capable of supporting pulmonary cells in tissue culture may be used.

In addition, the FPC culture medium can be supplemented with at least one growth factor. Preferably the growth factor is fibroblast growth factor (FGF). For example, any combination of FGF10, FGF7, FGF2 can be supplemented to the FPC culture medium. A preferred concentration of FGF7 is about 0.1-100 ng/ml (and any integer in between), more preferably the concentration is about 10 ng/ml. A preferred concentration of FGF10 is about 1-200 ng/ml (and any integer in between), more preferably the concentration is about 25 ng/ml. A preferred concentration of FGF2 is about 1-200 ng/ml (and any integer in between), more preferably the concentration is about 25 ng/ml.

Following isolation, FPCs are incubated in culture medium, in a culture apparatus for a period of time or until the cells reach confluency before passing the cells to another culture apparatus. Following the initial plating, the cells can be maintained in culture for a period of about 6 days to yield the Passage 0 (P0) population. The cells may be passaged for an indefinite number of times, each passage comprising culturing the cells for about 6-7 days, during which time the cell doubling time can range between about 3 to about 5 days. The culturing apparatus can be of any culture apparatus commonly used in culturing cells in vitro.

FPCs may be cultured in culture medium supplemented with FGF in the for a period of time or until the cells reach a certain level of confluence. Preferably, the level of confluence is greater than 70%. More preferably, the level of confluence is greater than 90%. A period of time can be any time suitable for the culture of cells in vitro. FPC culture medium may be replaced during the culture of FPCs at any time. Preferably, the culture medium is replaced every 3 to 4 days. FPCs are then harvested from the culture apparatus whereupon they may be used immediately or cryopreserved to be stored for use at a later time. FPCs may be harvested by trypsinization, EDTA treatment, or any other procedure used to harvest cells from a culture apparatus.

FPCs described herein may be cryopreserved according to routine procedures. Preferably, about one to ten million cells are cryopreserved in culture medium containing 10% DMSO in vapor phase of liquid N₂. Frozen cells may be thawed by swirling in a 37° C. bath, resuspended in fresh growth medium, and expanded as described above.

### Genetic Modification

Genetically modified FPCs are also useful in the instant invention. Genetic modification may, for instance, result in the expression of exogenous genes (“transgenes”) or in a change of expression of an endogenous gene. Such genetic modification may have therapeutic benefit. Alternatively, the genetic modification may provide a means to track or identify the cells so-modified, for instance, after implantation of a composition of the invention into an individual. Tracking a cell may include tracking migration, assimilation and survival of a transplanted genetically-modified cell. Genetic modification may also include at least a second gene. A second gene may encode, for instance, a selectable antibiotic-resistance gene or another selectable marker.

Proteins useful for tracking a cell include, but are not limited to, green fluorescent protein (GFP), any of the other fluorescent proteins (e.g., enhanced green, cyan, yellow, blue and red fluorescent proteins; Clontech, Palo Alto, Calif.), or other tag proteins (e.g., LacZ, FLAG-tag, Myc, His₆, and the like).

When the purpose of genetic modification of the cell is for the production of a biologically active substance, the substance will generally be one that is useful for the treatment of a given disorder. For example, it may be desired to genetically modify cells so that they secrete a certain growth factor product associated with bone or soft tissue formation. Growth factor products to induce growth of other, endogenous cell types relevant to tissue repair are also useful. For instance, growth factors to stimulate endogenous capillary and/or microvascular endothelial cells can be useful in repair of soft tissue defect, especially for larger volume defects.

The cells of the present invention can be genetically modified by having exogenous genetic material introduced into the cells, to produce a molecule such as a trophic factor, a growth factor, a cytokine, and the like, which is beneficial to culturing the cells. In addition, by having the cells genetically modified to produce such a molecule, the cell can provide an additional therapeutic effect to the mammal when transplanted into a mammal in need thereof. For example, the genetically modified cell can secrete a molecule that is beneficial to cells neighboring the transplant site in the mammal.

The FPCs may be genetically modified using any method known to the skilled artisan. See, for instance, Sambrook et al. (2001, Molecular Cloning: A Laboratory Manual, Cold Spring Harbor Laboratory Press, Cold Spring Harbor, N.Y.), and in Ausubel et al, Eds, (1997, Current Protocols in Molecular Biology, John Wiley & Sons, New York, N.Y.). For example, an FPC may be exposed to an expression vector comprising a nucleic acid including a transgene, such that the nucleic acid is introduced into the cell under conditions appropriate for the transgene to be expressed within the cell. The transgene generally is an expression cassette, including a polynucleotide operably linked to a suitable promoter. The polynucleotide can encode a protein, or it can encode biologically active RNA (e.g., antisense RNA or a ribozyme). Thus, for example, the polynucleotide can encode a gene conferring resistance to a toxin, a hormone (such as peptide growth hormones, hormone releasing factors, sex hormones, adrenocorticotrophic hormones, cytokines (e.g., interfering, interleukins, lymphokines), etc.), a cell-surface-bound intracellular signaling moiety (e.g., cell adhesion molecules, hormone receptors, etc.), a factor promoting a given lineage of differentiation (e.g., bone morphogenic protein (BMP)), etc.

Within the expression cassette, the coding polynucleotide is operably linked to a suitable promoter. Examples of suitable promoters include prokaryotic promoters and viral promoters (e.g., retroviral ITRs, LTRs, immediate early viral promoters (IEp), such as herpesvirus IEp (e.g., ICP4-IEp and ICP0-IEEp), cytomegalovirus (CMV) IEp, and other viral promoters, such as Rous Sarcoma Virus (RSV) promoters, and Murine Leukemia Virus (MLV) promoters). Other suitable promoters are eukaryotic promoters, such as enhancers (e.g., the rabbit .beta.-globin regulatory elements), constitutively active promoters (e.g., the .beta.-actin promoter, etc.), signal specific promoters (e.g., inducible promoters such as a promoter responsive to RU486, etc.), and tissue-specific promoters. It is well within the skill of the art to select a promoter suitable for driving gene expression in a predefined cellular context. The expression cassette can include more than one coding polynucleotide, and it can include other elements (e.g., polyadenylation sequences, sequences encoding a membrane-insertion signal or a secretion leader, ribosome entry sequences, transcriptional regulatory elements (e.g., enhancers, silencers, etc.), and the like), as desired.

The expression cassette containing the transgene should be incorporated into a genetic vector suitable for delivering the transgene to the cells. Depending on the desired end application, any such vector can be so employed to genetically modify the cells (e.g., plasmids, naked DNA, viruses such as adenovirus, adeno-associated virus, herpesviruses, lentiviruses, papillomaviruses, retroviruses, etc.). Any method of constructing the desired expression cassette within such vectors can be employed, many of which are well known in the art (e.g., direct cloning, homologous recombination, etc.). The choice of vector will largely determine the method used to introduce the vector into the cells (e.g., by protoplast fusion, calcium-phosphate precipitation, gene gun, electroporation, DEAE dextran or lipid carrier mediated transfection, infection with viral vectors, etc.), which are generally known in the art.

### Preparing Biocompatible Scaffolds

Scaffolds for use in the instant invention are made from biocompatible materials. The ideal properties of the biocompatible materials for use in the instant invention include at least one of: mechanical integrity, thermal stability, ability to self-assemble, non-immunogenic, bioresorbable, slow degradation rate, capacity to be functionalized with, for instance, cell growth factors, and plasticity in terms of processing into different structural formats.

Current scaffolds and tissue engineering techniques fail to permit long term maintenance of the differentiation state of alveolar epithelial cells (e.g., alveolar epithelial type II cells (AE2)) and branching morphogenesis in vitro. This is because in conventional methods, AE2 cells rapidly de-differentiate. However, the present invention provides a engineered three dimensional tissue that mimics natural lung tissue. The capability to create composites and scaffolds that mimic natural lung tissue enables the repair and regeneration of tissues and collections of tissues to a greater degree than prior art methods, and exhibits more accurate histological structure and function than can be achieved using prior art methods. For example, the engineered lung tissue comprises cells that exhibit budding structures and elongating tubular structures. Furthermore, the cells express genes involved in morphogenesis and lung epithelial differentiation. Non-limiting genes involved in morphogenesis and lung epithelial differentiation include distal epithelial marker genes SpC and SpB, the mesenchymal-derived morphogen FGF10, FGFr2, and vascular endothelial growth factor A (VEGF).

The physical characteristics of the composites and scaffolds is carefully considered when designing a substrate to be used in tissue engineering or repair. In order to promote tissue growth, the scaffold must have a large surface area to allow cell attachment. This is usually done by creating highly porous scaffolds wherein the pores are large enough such that cells can penetrate the pores. Furthermore, the pores can be interconnected to facilitate nutrient and waste exchange by the cells. These characteristics, i.e., interconnectivity and pore size, are often dependent on the method of fabrication.

An initial characteristic to consider when manufacturing composites and scaffolds is the choice of materials. It is understood that if the composites or scaffolds are manufactured for therapeutic use, all components used must be biocompatible. Accordingly, in considering substrate materials, it is imperative to choose one that exhibits clinically acceptable biocompatibility. In addition, the mechanical properties of the scaffold must be sufficient so that it does not collapse during the patient's normal activities. Both natural (e.g., collagen, elastin, poly(amino acids), and polysaccharides such as hyaluronic acid, glycosamino glycan, carboxymethylcellulose); and synthetic polymer materials may be used to manufacture the composites and scaffolds of the present invention. The polymer material may be in the form of one or more of sheet(s), blocks(s), pellets, granules, or any other desirably shaped polymer material.

The scaffold can include a number of biocompatible materials. The materials can include one or more of: collagens 1-9, glycoproteins, and attachment material such as fibronectin, laminin, thrombospondin, elastin, and fibrillin. Various matrix substances such as mucopolysaccharides, glycolipids, heparin sulfate, chondroitin sulfate, keratin sulfate, glycosaminoglycans, and hyaluronic acid can also be produced. The dynamic, living matrix, with its cells, can guide the development of new tissue formation by generating the needed matrix material essential to tissue and organ development. For example, if placed in the lung, the matrix will respond by making the essential pulmonary guiding material (e.g., FGF), which will allow branching morphogenesis. Without wishing to be bound by any particular theory, it is believed that when the biological matrix is implanted into the lungs, collagen will be synthesized as well as elastic fibers to provide the necessary basement membrane structure for epithelial cell attachment as well as the elastic expansion capabilities of the lung.

The biological matrix described herein can be used to form a scaffold by adding hydrogels or other materials that provide added shape, structure, or support. A variety of hydrogels can be used to prepare the new biological scaffolds. They include, but are not limited to: (1) temperature-dependent hydrogels that solidify or set at body temperature, (2) hydrogels cross-linked by ions, for example, sodium alginate; (3) hydrogels set by exposure to either visible or ultraviolet light, for example, polyethylene glycol polylactic acid copolymers with acrylate end groups; and (4) hydrogels that are set or solidified upon a change in pH.

The materials that can be used to form these various hydrogels include polysaccharides such as alginate, polyphosphazenes, and polyacrylates, which are cross-linked ionically, or block copolymers, which are poly(oxyethylene)-poly(oxypropylene) block polymers solidified by changes in temperature, or poly(oxyethylene)-poly(oxypropylene) block polymers of ethylene diamine solidified by changes in pH.

Once a hydrogel of choice (e.g., a thermosensitive polymer at between about 5 and 25% (w/v), or an ionic hydrogel such as alginate dissolved in an aqueous solution (e.g., a 0.1 M potassium phosphate solution, at physiological pH, to a concentration between about 0.5% to 2% by weight) is prepared, the biological matrix can be suspended in the polymer solution. The concentration of the cells can mimic that of the tissue to be generated. For example, the concentration of cells can range from between about 10 and 100 million cells/ml (e.g., between about 20 and 50 million cells/ml). Of course, the optimal concentration of cells to be delivered into the support structure can be determined on a case by case basis, and may vary depending on cell type and the region where the support structure is implanted or applied. To optimize the procedure (i.e., to provide optimal viscosity and cell number), one need only vary the amount of matrix or hydrogel.

The support structure is also biocompatible (i.e., it is not toxic to the cells suspended therein) and can be biodegradable. For example, the support structure can be formed from a synthetic polymer such as a polyanhydride, polyorthoester, or polyglycolic acid. The polymer should provide the support structure with an adequate shape and promote cell growth and proliferation by allowing nutrients to reach the cells by diffusion. Additional factors, such as growth factors, other factors that induce differentiation or dedifferentiation, secretion products, immunomodulators, anti-inflammatory agents, regression factors, biologically active compounds that promote innervation or enhance the lymphatic network, and drugs, can be incorporated into the polymer support structure. An example of a suitable polymer is polyglactin, which is a 90:10 copolymer of glycolide and lactide.

Alternatively, the polymer fibers can be compressed together in a mold that casts them into the shape desired for the support structure. In some cases, additional polymer can be added to the polymer fibers as they are molded to revise or impart additional structure to the fiber mesh. For example, a polylactic acid solution can be added to this sheet of polyglycolic fiber mesh, and the combination can be molded together to form a porous support structure. The polylactic acid can bind the crosslinks of the polyglycolic acid fibers, thereby coating these individual fibers and helping to fix the shape of the molded fibers. The polylactic acid can also fill in spaces between the fibers. Thus, porosity can be varied according to the amount of polylactic acid introduced into the support. The pressure required to mold the fiber mesh into a desirable shape can be quite moderate. All that may be required is that the fibers be held in place long enough for the binding and coating action of polylactic acid to take effect.

Alternatively, or in addition, the support structure can include other types of polymer fibers or polymer structures produced by techniques known in the art. For example, thin polymer films can be obtained by evaporating solvent from a polymer solution. These films can be cast into a desired shaped if the polymer solution is evaporated from a mold having the relief pattern of the desired shape. Polymer gels can also be molded into thin, permeable polymer structures using compression molding techniques known in the art.

Many other types of support structures are also possible. For example, the support structure can be formed from a sponge, foam, or biocompatible inorganic structure having internal pores, or from mesh sheets of interwoven polymer fibers. These support structures can be prepared using known methods.

Any of the natural scaffolding or liquid hydrogel-matrix mixtures described herein can be placed into any permeable support structure (also described herein). The scaffolding or liquid hydrogel-matrix mixture can be delivered to the shaped support structure either before or after the support structure is implanted into a patient. The specific method of delivery will depend on whether the support structure is sufficiently “sponge-like” for the given viscosity of the scaffolding or hydrogel-matrix composition, i.e., whether the support structure easily retains the biological scaffolding or liquid hydrogel-matrix mixture before it solidifies. Sponge-like support structures can be immersed within, and saturated with, the biological scaffolding or liquid hydrogel-matrix mixture, and subsequently removed from the mixture. The biological scaffolding or hydrogel is then allowed to solidify within the support structure. The biological scaffold- or hydrogel-matrix-containing support structure can then be implanted in or otherwise applied to the patient.

A preferred model for use in the present invention comprises a collagen gel, which may be formed, for example, formed from a solution of collagen into which FPCs are mixed. Once it has set, the FPCs contract the gel into a connective tissue-like scaffold. The collagen is preferably Type I collagen, Type III collagen, or a combination of the two. The collagen solution from which the gel is formed preferably has a collagen concentration of between 0.3 mg/ml and 3.0 mg/ml collagen.

This protocol has the advantage of providing a matrix which mimics that occurring in vivo, without the use of non-physiological substrates or supports such as nylon mesh, used in other tissue modelling constructs. In the present invention, the cells are incorporated directly into a contracted gel formed from collagen, which is the major natural component of tissue matrix, and provides a much more physiologically relevant model of the interactions between the cells and the underlying tissue.

Further components found in physiological connective tissue may be added to the collagen gel as desired. These ray include molecular components such as hyaluronic acid and chondroitin sulphate.

The scaffold is responsive to external micro-environmental tissue cues, and this responsiveness can provide the essential type of matrix structure and environment conducive to the precise matrix guidance of tissue construction. For example, the pattern of collagen, basement membrane, reticular fibers, or laminin can be synthesized by the spore-like cells. These structures provide guidance for the organization of tissue including the attachment of tissue to the matrix. The synthesis of these guidance structures can occur in concert with the synthesis of other essential structures, such as basement membrane. The synthesis of basement membrane provides for epithelial attachment and interaction with mesenchymal connective tissue, and also allows for the ingrowth of blood vessels.

To obtain the cells and the materials necessary to generate a new biological scaffolding, a piece of tissue from a donor can be placed in a buffered solution (e.g., phosphate buffered saline), which can include one or more antibiotics, and the tissue can be dissociated mechanically (e.g., by macerating the tissue), chemically (e.g., by exposure to one or more enzymes, such as trypsin or collagenase, that facilitate tissue degradation), or both.

The invention also provides cells that “seed” the scaffold. FPCs can be cultured on the scaffold. The cells can also differentiate in vitro by culturing the cells in differentiation medium. Alternatively, the cells can differentiate in vivo when they establish contact with a tissue within the mammal or when the cells are sufficiently close to a tissue to be influenced by substances (e.g., growth factors, enzymes, or hormones) released from the tissue. In other words, FPCs of the matrix can establish contact with a tissue, such as lung, by virtue of receiving signals from the tissue. Such signaling would occur, for example, when a receptor on the surface of a FPC, or on the surface of a cell descended from a FPC, binds and transduces a signal from a molecule such as a growth factor, enzyme, or hormone that was released by a tissue within the mammal. These agents guide differentiation so that the FPCs come to express some and possibly most (if not all) of the same proteins normally expressed by differentiated cells in the tissue in which they have been placed.

Alternatively, or in addition, FPCs of the matrix can be induced to differentiate by adding a substance (e.g., a growth factor, enzyme, hormone, or other signaling molecule) to the cell's environment. For example, a substance can be added to the biological scaffolding of the invention.

While FPCs and associated cellular matrix can eventually become fully differentiated, and while this is desirable in some circumstances (e.g., where the cells are used to recreate a histologically mature and complete tissue), not all of the cells administered need to be fully differentiated to achieve successful treatment; FPCs of the cellular matrix need only differentiate to a point sufficient to treat the mammal. That point can be reached either before or after the matrix is administered to the patient.

Differentiation occurs when a cell of the matrix expresses essentially the same phenotype as a mature cell at the site of implantation. For example, for the purpose of defining this invention, a FPC of a cellular matrix, having been implanted into the lung, is differentiated when it expresses essentially the same proteins expressed by the lung, e.g., an alveolar epithelial cell. Antibodies to lung markers are commercially available or otherwise readily attainable.

Differentiated cells can also be identified by their gross morphology and by the connections they form with other cells. For example, cells that differentiate into lung cells can develop complex morphology resembling bronchioles. For example, the invention is based on the novel discovery that culturing FPCs on a three dimensional scaffold resulted in the induction of branching morphogenesis and sacculation which corresponded with the expression of surfactant protein C (AE2 marker), FGF10 (mesenchymal-derived morphogenetic inducer of the epithelium), and FGFr 2 (epithelial morphogenetic receptor).

### Administration

The invention also provides methods of treating a patient by implanting a biological matrix comprising FPCs in the presence or absence of a scaffold into a tissue of the patient, such as the lung. After implantation, the grafted cells can respond to environmental cues that will cause it to develop characteristics of the endogenous tissue. For example, if the cells are implanted into lung tissue, it will be induced to synthesize a collagen and/or an elastic fiber. Preferably, the cells form histiotypic alveolar-like structures, comprised of differentiated distal epithelial cells (proSpC expressing) forming ductal structures. Thus, the implanted cells will develop characteristics that liken it to the surrounding tissue. By these methods, the biological scaffolding can augment the tissue; the biological scaffolding of the invention can be used for tissue engineering and in any conventional tissue engineering setting.

The biological matrix can be administered directly, without any support structures. For example, the matrix can be suspended in a physiologically compatible solution and injected into an organ or tissue. For example, the matrix can be applied directly by syringe and needle or micro-catheter to an area of tissue that has been damaged or adversely affected by disease. Development of the FPCs enmeshed in the injected matrix will be driven by factors in the local environment and will replenish and repopulate the area.

Accordingly, the invention encompasses tissue regeneration applications. The objective of the tissue regeneration therapy approach is to deliver high densities of repair-competent cells (or cells that can become competent when influenced by the local environment) to the defect site in a format that optimizes both initial wound mechanics and eventual neotissue production. The composition of the instant invention is particularly useful in methods to alleviate or treat lung tissue defects in individuals. Advantageously, the composition of the invention provides for improved lung tissue regeneration. Specifically, the tissue regeneration is achieved more rapidly as a result of the inventive composition.

The composition of the invention may be administered to an individual in need thereof in a wide variety of ways. Preferred modes of administration include intravenous, intravascular, intramuscular, subcutaneous, intracerebral, intraperitoneal, soft tissue injection, surgical placement, arthroscopic placement, and percutaneous insertion, e.g. direct injection, cannulation or catheterization. Most preferred methods result in localized administration of the inventive composition to the site or sites of tissue defect. Any administration may be a single application of a composition of invention or multiple applications. Administrations may be to single site or to more than one site in the individual to be treated. Multiple administrations may occur essentially at the same time or separated in time.

Advantageously, the compositions and methods of the invention improve on prior art methods. Preferably the composition for use in treating a lung tissue defect comprises FPCs, more preferably FPCs seeded on a scaffold and cultured in vitro in the presence of FGF, 3-dimensional culture conditions, as described elsewhere herein.

### Model for Drug Discovery

The present invention provides an in vitro method suitable to allow evaluation of test compounds for therapeutic activity with respect to a pulmonary disease or disorder. Preferably, the method includes the use of an engineered three dimensional lung tissue.

The invention is based on a model developed using FPCs. In some instances, mixed populations of FPC which contain epithelial, mesenchymal, and endothelial cells are used to generate the three dimensional lung tissue. For example, the FPCs are placed within a three dimensional collagen gel that mimics a connective tissue matrix. Thus, the model incorporates the influence of FPC on the growth and cell-cell communication with neighboring cells. The three dimensional lung tissue mimics a natural lung tissue, for example the engineered lung tissue exhibits branching morphogenesis exemplified by natural lung tissue.

The model is useful for testing drugs on the pathology of a lung tissue. In addition, the model can be used to examine the effects of particular delivery vehicles for therapeutic agents on the pathology of lung tissue, for example, to compare the effects of the same agent administered via different delivery systems, or simply to assess whether a delivery vehicle itself (e.g. a viral vector) is capable of affecting lung pathology.

In one embodiment, the invention provides an in vitro method for screening a test agent for the ability of the test agent to modulate the health of a lung tissue. The method comprises contacting a test agent to an engineered three dimensional lung tissue model and measuring the effect that the test agent has on the lung tissue model. Any alteration to the model in the presence of the test agent is an indication that the test agent is able to modulate the health of a lung tissue.

In another embodiment, the present invention provides an in vitro method for observing an effect a test agent has on a lung tissue, comprising the steps of:
a) providing at least one three-dimensional lung tissue model, wherein the model is intended to model normal lung tissue;
b) contacting the test agent with the lung tissue model; and
c) observing the effect the test agent has the lung tissue model.

The tissue model is a construct which comprises a three-dimensional array of cells on a scaffold, for example a collagen matrix, and at least one test cell. The method comprises observing the effect of the test agent on the pathology of the lung tissue. However the method may further comprise the step of observing the effect of the test agent on individual cell types of the lung tissue.

The test agent may be any agent including chemical agents (such as toxins), pharmaceuticals, peptides, proteins (such as antibodies, cytokines, enzymes, etc.), and nucleic acids, including gene medicines and introduced genes, which may encode therapeutic agents such as proteins, antisense agents (i.e. nucleic acids comprising a sequence complementary to a target RNA expressed in a target cell type, such as RNAi or siRNA), ribozymes, etc. Additionally or alternatively, the test agent may be a physical agent such as radiation (e.g. ionising radiation, UV-light or heat); these can be tested alone or in combination with chemical and other agents.

The model may also be used to test delivery vehicles. These may be of any form, from conventional pharmaceutical formulations, to gene delivery vehicles. For example, the model may be used to compare the effects on a therapeutic effect of the same agent administered by two or more different delivery systems (e.g. a depot formulation and a controlled release formulation). It may also be used to investigate whether a particular vehicle-could have effects of itself on the lung tissue. As the use of gene-based therapeutics increases, the safety issues associated with the various possible delivery systems become increasingly important. Thus the models of the present invention may be used to investigate the properties of delivery systems for nucleic acid therapeutics, such as naked DNA or RNA, viral vectors (e.g. retroviral or adenoviral vectors), liposomes, etc. Thus the test agent may be a delivery vehicle of any appropriate type with or without any associated therapeutic agent.

The test agent may be added to said model to be tested by any suitable means. For example, the test agent may be added drop-wise onto the surface of the model and allowed to diffuse into or otherwise enter the model, or it can be added to the nutrient medium and allowed to diffuse through the collagen gel. The model is also suitable for testing the effects of physical agents such as ionising radiation, UV-light or heat alone or in combination with chemical agents (for example, in photodynamic therapy).

Observing the effect the test agent has on said models may include a variety of methods. For example, a particular agent may induce a cell to enter apoptosis. Detectable changes in the cell may comprise changes in cell area, volume, shape, morphology, marker expression (e.g. cell surface marker expression) or other suitable characteristic, such as chromosomal fragmentation. Cell number may also be monitored in order to observe the effects of a test agent on cell proliferation; this may be analysed directly, e.g. by counting the number of a particular cell type present, or indirectly, e.g. by measuring the size of a particular cell mass. These may be observed directly or indirectly on the intact model using, for example, suitable fluorescent cell staining. This can be by pre-labelling of cells with vital dyes or genetically introduced fluorescent markers (for example green fluorescent proteins) for serial analysis of the living model or by fixation and post-labelling with fluorescent substances such as propidium iodide or fluorescently labelled antibodies. Alternatively, models may be processed by normal histological methods, such as immunohistochemistry, using antibodies directed against a suitable cellular target, or in situ hybridization, to test for expression of a particular mRNA species. Moreover, this may be carried out in an automated/robotic or semi-automated manner, using computer systems and software to image the cells at various time points and detect any change in, for example, cell density, location and/or morphology. Confocal laser scanning microscopy in particular permits three-dimensional analysis of intact models. Thus it is possible to apply directly to the intact, three-dimensional lung tissue model, quantitative analysis of cell behavior which are normally only possible for cells in conventional two-dimensional culture. By this means quantitative, serial analysis of cell proliferation, apoptosis, necrosis, migration and matrix invasion, among others, are obtained in a three-dimensional lung tissue model which bridges the gap between conventional two-dimensional cell cultures and live animal models.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Effects of FGF Fetal Pulmonary Cells (FPC) Cultured in 3-D Collagen Gels

The following experiments were designed to assess the effects of exogenous fibroblast growth factors, FGF10, FGF7 and FGF2, on mixed populations of embryonic day 17.5 murine fetal pulmonary cells (FPC) cultured in 3-D collagen gels in the context of forming an engineered lung tissue.

The morphogenic effects of the FGFs alone and in various combinations were assessed by whole mount immunohistochemistry and confocal microscopy. It was observed that the combination of FGF10 and FGF7 significantly increased epithelial budding and proliferation whereas FGF10 alone induced widespread budding. FGF7 alone induced dilation of epithelial structures, but not widespread budding. FGF2 alone had a similar dilation as compared to FGF7, but not budding. In addition, FGF2 had a similar effect in epithelial structures, and also significantly enhanced endothelial tubular morphogenesis and network formation, as well as mesenchymal proliferation. The combination of FGF10/7/2 induced robust budding of epithelial structures and the formation of uniform endothelial networks in parallel.

Without wishing to be bound by any particular theory, it is believed that appropriate combinations of exogenous FGFs chosen to target specific FGFR isoforms allow for control of lung epithelial and mesenchymal cell behavior in the context of an engineered system. It is believed that tissue engineered fetal distal lung constructs provide a potential source of tissue or cells for lung augmentation in pediatric pulmonary pathologies, such as pulmonary hypoplasia and bronchopulmonary dysplasia. In addition, engineered provide alternative in vitro venues for the study of lung developmental biology and pathobiology.

The materials and methods employed in these experiments are now described.

### Materials and Methods

Fetal Pulmonary Cell Isolation and In Vitro Culture

Embryonic day 17.5 (E17.5) murine fetal pulmonary cells (FPC) were obtained from the lungs of timed-pregnant Swiss Webster mouse fetuses (Charles River Laboratories), according to an approved protocol (IACUC #30511), essentially as previously described (Mondrinos, et al., 2006, Tissue Eng 12(4): 717-28). Following initial isolation, the FPC were centrifuged and resuspended in a 1.2 mg/ml liquid collagen solution (BD Biosciences) at physiological pH, at a density of 2.5-5.0 million FPC/ml. One milliliter of cell/collagen mixture per well were cast in 24 well plates and transferred to the incubator. Following polymerization of the gel, 2 ml of an 80:20 mixture of DMEM/F12 medium (Cambrex) containing 10% fetal bovine serum (Hyclone), L-glutamine, and penicillin-streptomycin antibiotics was overlaid and the constructs were incubated overnight. Subsequently, the constructs were maintained in 2 ml serum-free basal DMEM/F12 medium supplemented with 1% insulin-transferrin-selenium (1% ITS, BD Biosciences) and heparin (Sigma) (10 units/ml); 10% FBS, or 1% ITS supplemented with FGF7 (10 ng/ml), FGF10 (25 ng/ml) or FGF2 (25 ng/ml) alone or in combination as follows: FGF10; FGF7; FGF2; FGF10/7; FGF10/7/2. All cell culture was carried out at 37° C. in a 5% CO₂ humidified incubator. The medium was replaced every 48 hours for the first week, then every 24 hours for cultures that were extended to 14 days.

Whole Mount Immunohistochemistry

Morphologic and phenotypic characterization of the in vitro constructs was carried out utilizing a whole mount indirect fluorescent immunohistochemistry (IHC) protocol similar to that used for whole mount staining of embryos and explants (Sillitoe, et al., 2002, J Histochem Cytochem 50(2):235-44; Snow, et al., 2005, Anat Rec A Discov Mol Cell Evol Biol 282(2): 95-105). In brief, 3-D constructs were fixed in 4% paraformaldehyde (Electron Microscopic Sciences) for 1 hour at room temperature and then overnight at 4° C. and washed 3×20 minute in 1× tris-buffered saline (TBS) containing 100 mM glycine (Sigma) pH=7.4, to reduce background autofluorescence. All steps were performed at room temperature on a benchtop orbital shaker (Belly Dancer, Stovall). Constructs were washed briefly in 1× TBS and then permeabilized/blocked using 0.5% triton-X and 3% BSA in 1× TBS for 6-8 hours.

Following the permeabilization and blocking, constructs were washed 3×5 minute in 1× TBS with 1% BSA. Constructs were then incubated with either polyclonal rabbit primary antibodies against pan-cytokeratin to visualize the intermediate filaments in all epithelial cells (Dako, 1:100), prosurfactant protein C to identify type II alveolar epithelial cells (Chemicon, 1:100), PECAM-1 (Abcam, 1:50) to identify endothelial cells, and tropoelastin (Abcam, 1:100), as a marker for mesenchymal cells. All primary antibodies were prepared in 1× TBS containing 0.1% triton-X and 1% BSA. Negative controls were processed identically, except that the specific primary antibodies were replaced with normal rabbit IgG (1:50-1:100). After washing 3×1 minute with 1× TBS, the constructs were washed 3×20 minutes in 1× TBS with 1% BSA, then for 2 hours in a large volume (15 ml tube for each sample) of 1× TBS. Samples were then washed once more with 1× TBS+3% BSA+0.2% triton-X for 30 minutes prior to secondary antibody application. Secondary antibodies, fluorescent goat anti-rabbit IgGs (Alexa488 or Alexa594, Invitrogen), were prepared at dilutions of 1:500 in 1× TBS containing 0.1% triton-X and 1% BSA and incubated with constructs for 2 hours.

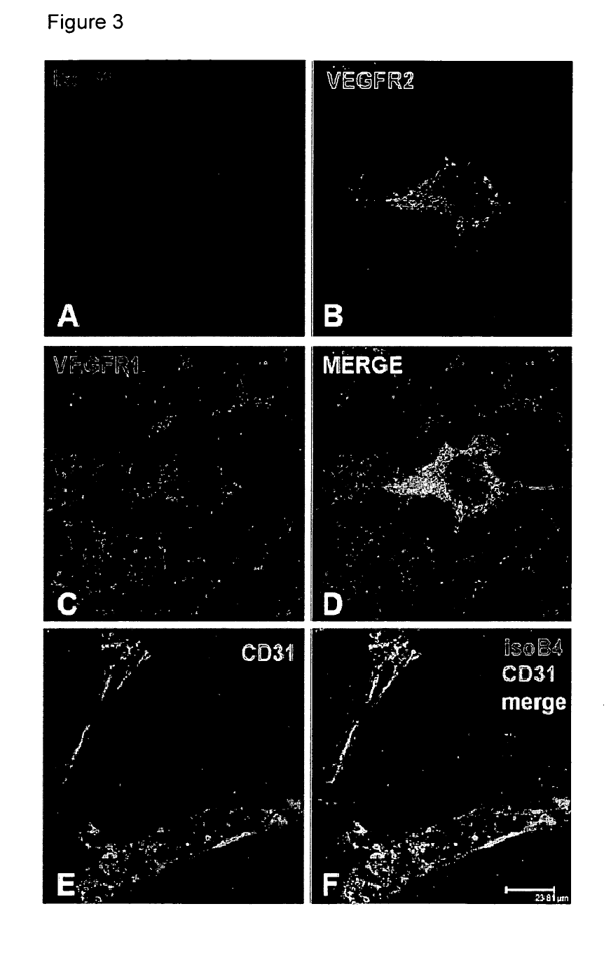

Endothelial cells were identified by staining with Griffonia simplicifolia lectin I—isolectinB4 (isoB4) (Invitrogen). Depending on the multi-staining protocol, isoB4 was used conjugated to either Alexa488, Alexa568, or Alexa647, respectively. The endothelial specificity of isoB4 has been reported previously (Akeson, et al., 2005, Pediatr Res. 57(1): 82-8; Hyink, et al., 1996, Am J Physiol 270(5 Pt 2): F886-99; Laitinen, 1987, Histochem J 19(4):225-34) and was also confirmed in the experiments disclosed herein (FIG. 3). For multiplex immunocytochemistry of VEGFRs and FGFRs, commercially available kits (Zenon™ anti-rabbit Alexa dye labeling kits) were used to generate fluorescent conjugates of rabbit polyclonal antibodies against VEGFR1 and VEGFR2 (Neomarkers) and FGFR1 and FGFR2 (Abgent) according to manufacturer instructions. Primary fluorescent antibody conjugates were used at 1:50 dilutions for 30 minutes. Staining patterns were confirmed by comparison to single target indirect immunofluorescence in separate experiments. When double staining with isoB4 was performed, a 10 μg/ml solution of the desired isoB4 conjugate was prepared and admixed to either the secondary antibody solution, or along with the primary conjugates used for multiplex immunocytochemistry.

Constructs were washed 3×20 minutes with 1× TBS, then for 2 hours in a large volume of 1× TBS (15 ml tube for each sample) prior to mounting with anti-fade medium (Vectashield, Vector Labs) and visualization by laser scanning confocal microscopy (Leica). Digital images were acquired using proprietary software from Leica for conventional and confocal microscopy, respectively. 3-D z-projections of whole mount staining were generated using the Leica confocal software.

Quantitative Image Analysis and Statistical Analysis

Quantitative analysis of phase contrast images of alveolar forming units (AFUs) taken at 7 days for epithelial morphometry was carried out using NIH ImageJ. Images were all taken at 100× magnification. For each sample/condition/experiment a minimum of 10 images containing about 25 individual AFUs were analyzed. Individual AFUs were manually outlined using the region of interest (ROI) selection tool. Once selected, the area of individual AFUs (pixels) was measured. Normalized areas were calculated for each independent experiment, setting 1% ITS equal to 1. Normalized mean areas for each independent experiment were then averaged to yield a cumulative value. The data are represented as fold increase over 1% ITS. Rudimentary bud counts for individual AFUs were performed manually in parallel with area measurements and the results were normalized to 1% ITS in a similar fashion. Statistical analysis of the area measurements and bud counts were carried out by one-way ANOVA with the Tukey post-test (T-test) for individual comparisons between area values for the various media supplementation conditions.

Quantification of isoB4 staining in laser scanning confocal micrographs was carried out using NIH ImageJ. For each experimental condition, at least 20 randomly acquired 200× fields was analyzed at comparable z-positions taken from at least 2 whole mount constructs. Individual images were binarized, and total area of isoB4 stained pixels per 200× microscopic field was calculated. Using the same data, a morphogenetic index, termed the index of elongation and interconnectivity was determined by measuring the fraction of total area of isoB4 staining contributed by interconnected/elongated EC area vs. single EC (Index=Area of interconnected EC/(Area of interconnected EC+Area of single EC)). These values are basically zero for 1% ITS and 10% FBS cultures. Statistical analysis of the area measurements was carried out by one-way ANOVA with the Tukey post-test (T-test) for individual comparisons between area values for the various media supplementation conditions. P values were calculated by Student's t-test with p<0.05 being regarded as statistically significant.

Reverse Transcriptase Polymerase Chain Reaction (RT-PCR)

RT-PCR was utilized to detect steady state mRNA expression of relevant genes in 3-D collagen gel constructs as previously reported for 3-D Matrigel (Mondrinos, et al., 2006, Tissue Eng 12(4): 717-28). In brief, collagen constructs were digested and RNA extracted with TriReagent™ (Sigma) according to published protocols (Mondrinos, et al., 2006, Tissue Eng 12(4): 717-28). RT-PCR was performed using a commercially available kit (Promega) following the manufacturer's instructions. Primers for surfactant proteins B and C (SpB and SpC), FGF10, vascular endothelial growth factor A (VEGF), and glyceraldehyde-3-phosphate dehydrogenase (GAPDH), were obtained from Quiagen based on the Clontech Atlas™ (Mouse 1.2 Array II, Cat. #7857-1, BD Biosciences). For all genes a 30 cycle PCR routine was used as previously described (Mondrinos, et al., 2006, Tissue Eng 12(4): 717-28). Total RNA isolated from E17.5 fetal pulmonary tissue was used as a positive control. Negative controls included no reverse transcription samples, as well as reactions without the addition of the cDNA template.

Viability Staining

Cell viability was assessed at 7 days in select experiments by using the LiveDead kit (Invitrogen). Briefly, following removal of cell culture medium, 1 ml of 2 μM ethidium homodimer and 4 μM calcein-AM in 1× PBS was added to the constructs, which were then incubated for 30-45 minutes at room temperature on an orbital shaker. Samples were then washed with 1× PBS (3×5 minutes) and immediately imaged on a fluorescent microscope (Leica). Imaging was delicate, as the unfixed samples were fragile. Photobleaching of the calcein-AM during focusing in the 3-D gels was also problematic. Nevertheless, differences in the viability of cells in constructs cultured with the various media were clearly discernible.

The results of the experiments are now described.

### Effect of FGF Supplementation on Epithelial Morphogenesis and Cytodifferentiation

The results presented herein demonstrate the successful in vitro formation of histiotypic 3-D lung alveolar constructs in Matrigel hydrogels. Epithelial structures within these constructs, termed alveolar forming units (AFUs), were multicellular, lumen-containing assemblies which branched differentially depending on media composition. The following experiments were designed to quantitatively analyze by way of phase contrast micrographs the AFUs present in collagen gel constructs generated with various FGF media compositions in terms of a) AFU area, as a measure of epithelial growth, and b) rudimentary bud counts as a measure of epithelial morphogenesis.

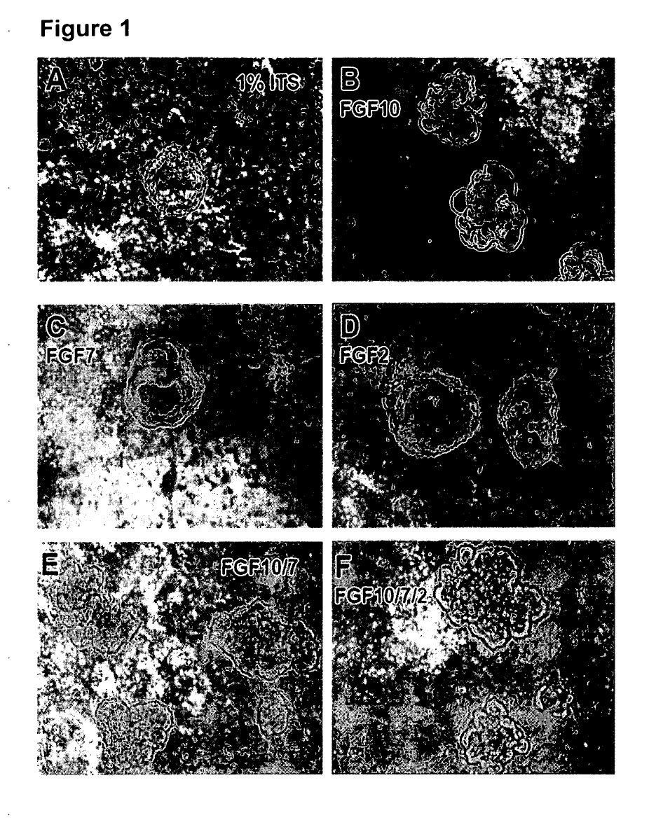

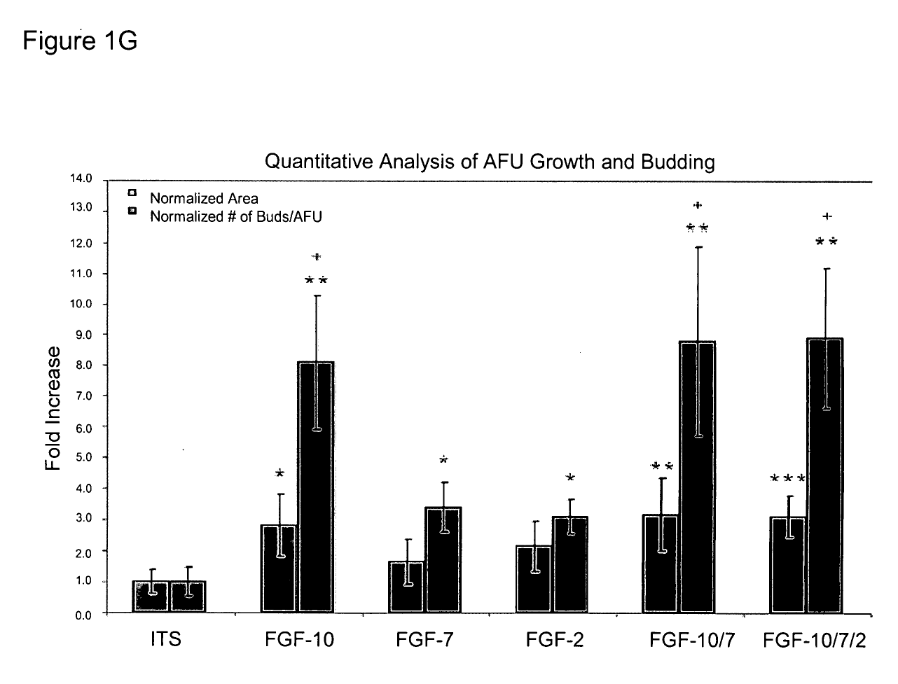

Constructs cultured in basal medium supplemented with only 1% ITS (FIG. 1A) had the lowest levels of epithelial growth and bud formation (FIG. 1G); all subsequent quantitative data are normalized to this baseline condition. Of all the single fibroblast growth factor additions to the medium, only addition of FGF10 resulted in a statistically significant (about 3 fold) increase in AFU area, (FIG. 1G). In terms of histiotypic branching morphogenesis, the architecture of AFUs formed in FGF7 and FGF2 only conditions showed a significant, yet modest increase in rudimentary bud counts (about 3 fold increase over 1% ITS), however this increase was significantly less than the about 8 fold increase in the number of buds/AFU induced by FGF10. AFUs growing in FGF7 (FIG. 1C) and FGF2 (FIG. 1D) were dilated compared to 1% ITS and exhibited a cystic architecture, without widespread bud formation. Co-supplementation of FGF10/7 did not result in statistically significant increases in AFU area or numbers of buds/AFU, however the structures appear more dilated than in FGF10 only cultures (FIG. 1E vs. 1B). FGF10/7/2 did not further enhance growth or budding of AFUs compared to FGF10/7 or FGF10 cultures (FIGS. 1F, 1G).

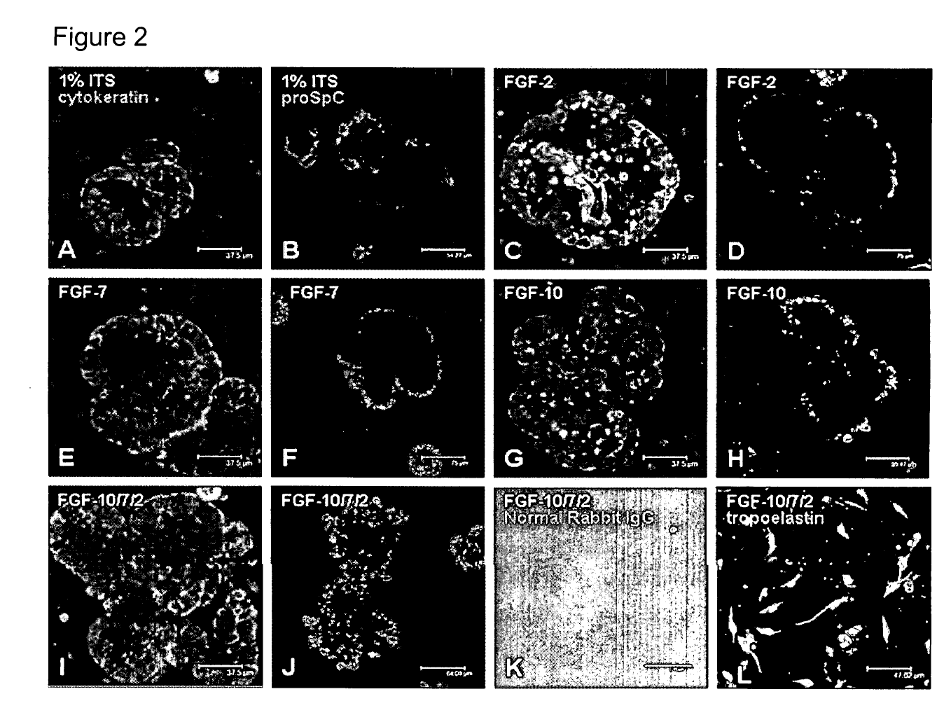

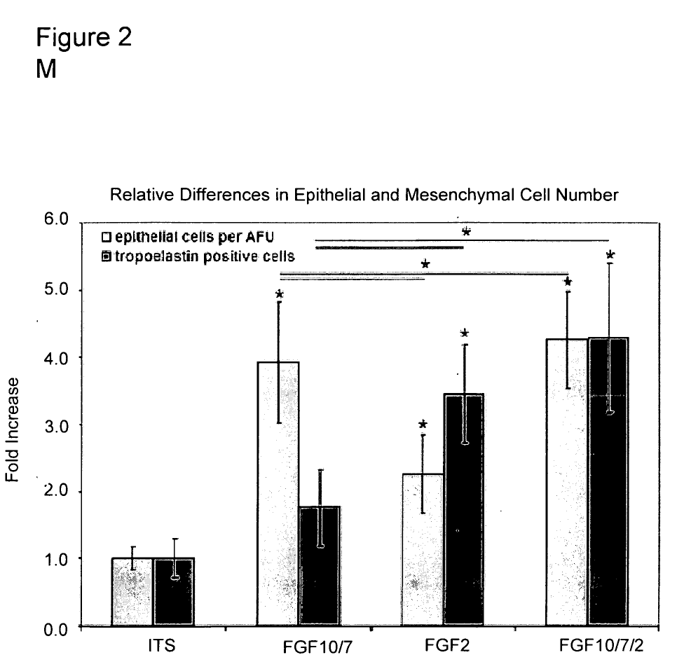

The architecture and epithelial differentiation of AFUs under various conditions was further analyzed by confocal microscopy in optical sections of whole mounts stained for cytokeratin and prosurfactant protein C (proSpC). Negative controls were stained with normal rabbit IgG antibodies (FIG. 2K). In the presence of 1% ITS only, small circular structures of epithelial cells, without bud formation (FIG. 2A) and sparse proSpC immunoreactivity (FIG. 2B) were observed. Culture with 10% FBS yielded the formation of slightly larger, more solid aggregates of epithelial cells with similar sparse proSpC immunoreactivity (data not shown). Addition of FGF7 and FGF2 yielded largely circular AFUs with cystic morphology that displayed patchy, but consistent, proSpC reactivity (FIGS. 2C-2F). Supplementation with FGF10 alone induced a histiotypic budding architecture of the epithelial cells (FIG. 2G), which correlated with more uniform, intense proSpC staining in the cells lining these structures (FIG. 2H). This increased localization of proSpC in AFUs was even more pronounced in cultures supplemented with either FGF10/7 (data not shown) or FGF10/7/2 (FIG. 2J).

In line with previous reports (Bruce, et al., 1998, Am J Physiol. 274(6 Pt 1): L940-50; Nakamura, et al., 2000, Am J Physiol Lung Cell Mol Physiol. 278(5): L974-80), tropoelastin was used as a marker for identifying mesenchymal cells and for evaluating how exogenous FGFs might affect mesenchymal proliferation. Tropoelastin positive cells, with fibroblastic morphology were present in the interstitial spaces of all constructs (FIG. 2L). By counting the numbers of epithelial nuclei in cytokeratin stained AFUs and tropoelastin positive cells in the interstitial spaces, differential affects of FGF10/7 and FGF2 on epithelial and mesenchymal cell numbers in the constructs was assessed. FGF10/7 induced a statistically significant ˜4 fold increase in the number of epithelial cells per AFU (FIG. 2M), which correlates well with the ˜3 fold increases in AFU area measured in phase contrast images (FIG. 1G). By contrast FGF10/7 produced a more modest 1.5-2 fold increase in numbers of tropoelastin-positive cells. A ˜2 fold increase in epithelial cell number per AFU (FIG. 2M), similar to AFU area measurements (FIG. 1G) was induced by FGF2. At the same time, however, FGF2 induced ˜3.5 fold increase in numbers of tropoelastin-positive cells. FGF10/7/2 induced a ˜4 fold increase in both epithelial cells per AFU and tropoelastin-positive cell numbers (FIG. 2M), apparently additively combining the separately observed effects of FGF10/7 and FGF2.

### Effect of FGF Supplementation on Endothelial Morphogenesis

Based on periodic counting of isolectinB4+ endothelial cells (ECs) in overnight (12-16 hour) cultures of freshly isolated FPC, it is believed that the FPC preparation contain approximately 20-30% ECs (data not shown). The endothelial phenotype of isolectinB4 positive cells was independently confirmed by immunofluorescence staining for VEGFR1 (Flt1) and VEGFR2 (KDR) in combination with isolectinB4 labeling (FIGS. 3A-3D). In line with previous reports of VEGFR expression in fetal lung mesenchymal cells, VEGFR2 staining was restricted to isolectinB4+ ECs (FIG. 3B), while VEGFR1 staining was observed both in isolectinB4+ ECs and in most other cells of apparent mesenchymal nature (FIG. 3C). In 3-D gels, upon FGF supplementation, ECs formed tubular networks as visualized by PECAM-1 staining (FIG. 3E). IsolectinB4 staining was more uniform (FIG. 3F) and was therefore utilized for subsequent analysis, specifically for the 3-D visualization of EC tubular morphogenesis in the constructs.

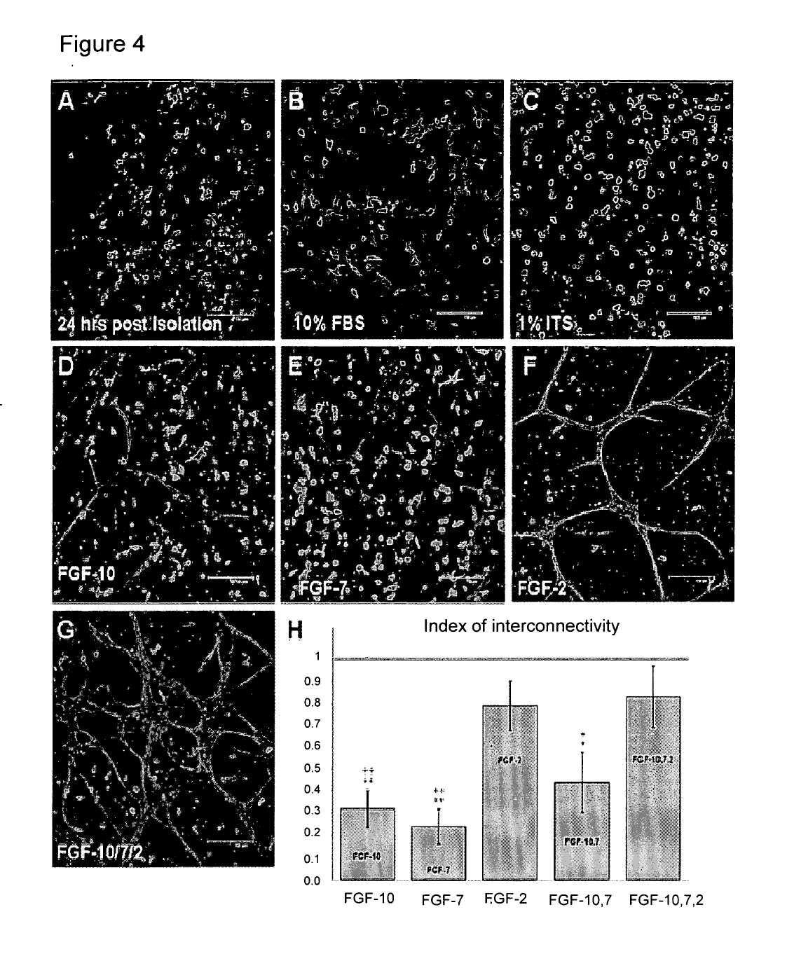

3-D z-projections of isolectinB4 stained constructs revealed relatively uniform distribution of single EC within the first 24 hours post-seeding (FIG. 4A). Following 7 days of culture, all samples, with the exception of 10% FBS and 1% ITS, consistently contained elongated and interconnected ECs (FIGS. 4B and 4C). In order to quantify the degree of EC network assembly, an index of interconnectivity was calculated. This value was virtually zero for constructs 24 hours post-seeding, as well as constructs maintained for 7 days in 10% FBS or 1% ITS, as also confirmed visually (FIGS. 4A-C). The addition of any single FGF to the medium consistently yielded elongated and interconnected ECs by day 7. In the cases of FGF10 (FIG. 4D), FGF7 (FIG. 4E), and FGF10/7 (data not shown) a few elongated, interconnected EC networks were observed; however there were also many single ECs. The index of interconnectivity analysis for FGF10, FGF7 and FGF10/7 yielded values ranging from about 0.2-0.4, however these values were not statistically different from each other (FIG. 4H). Widespread, relatively uniform formation of EC networks comprising largely interconnected tubular EC assemblies was observed in the case of FGF2 (FIG. 4F) and FGF10/7/2 (FIG. 4G), as also reflected by an index of interconnectivity of ˜0.8 (FIG. 4H). Despite the similar index of interconnectivity values for FGF2 and FGF10/7/2, the networks formed in FGF10/7/2 cultures (FIG. 4G) were more complex, with more and thicker branch points, and consequently more connecting tubules compared with FGF2 cultures (FIG. 4F). Preliminary measurements of network complexity in digitally skeletonized 3-D projections of endothelial networks in FGF2 vs. FGF10/7/2 cultures, indicated an approximate two fold increase in the number of branch points and the total tubule length in the presence of FGF10/7/2, as compared to FGF2 alone (data not shown).

Epithelial-Endothelial Interfacing with FGF Supplementation

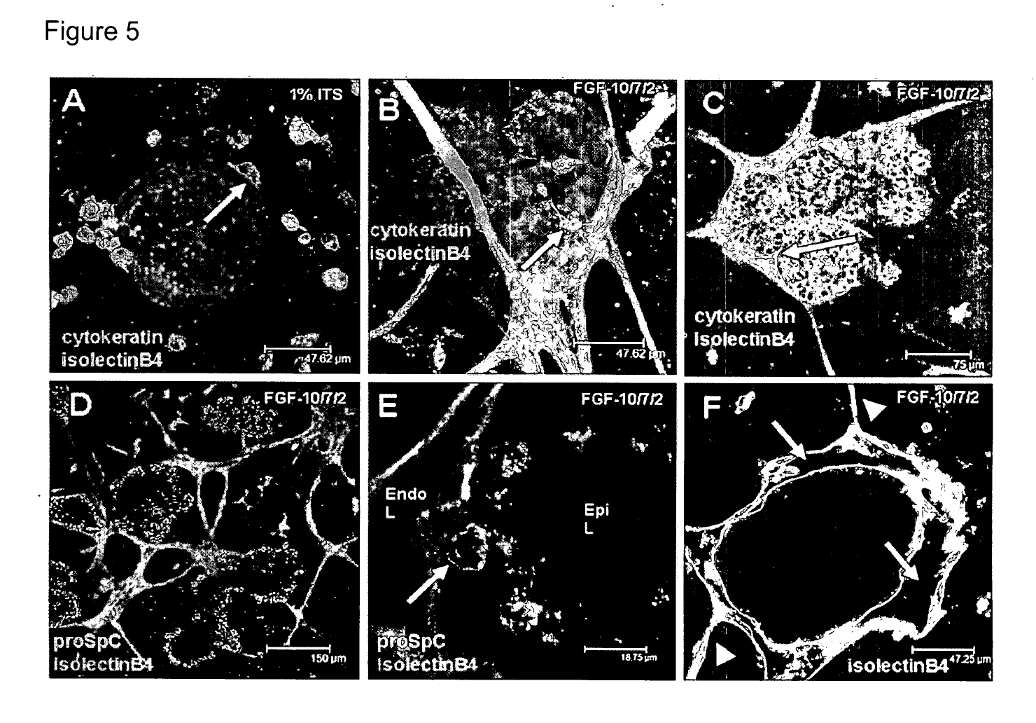

The epithelial-endothelial interface in developing AFUs was visualized by double staining for endothelial cells (isolectinB4) and epithelial cells (cytokeratin or proSpC). Various fluorophore combinations were used and the marker associated with each color is clearly labeled in the panels of each figure. Under serum-free, baseline conditions (1% ITS), most ECs remained non-elongated rounded, single cells, however some interfacing between endothelial cells and epithelial cells was observed (arrow in FIG. 5A). In the case of FGF10/7/2 supplementation, AFUs were tightly interfaced with and enrobed by interconnected tubular structures comprised of ECs (FIG. 5B). Penetration of EC capillary-like structures into the clefts of AFUs between neighboring epithelial buds was widely observed in FGF10/7/2 cultures (FIG. 5B arrow and FIG. 5C arrow). ProSpC staining illustrates the alveolar type II epithelial nature of nearly all the cells comprising AFUs enrobed by endothelial networks in the FGF10/7/2 condition (FIG. 5D). FIG. 5E illustrates the interfacing of proSpC expressing cells comprising bud structures and lumenized endothelial structures (FIG. 5E arrow). Endothelial tubules remote from developing AFUs often appeared as “endothelial cords” that did not contain lumina, however in areas directly contacting epithelium; consistent lumen formation was observed (FIGS. 5E and 5F). Shown in FIG. 5F are isolectinB4+ ECs interfaced directly with an epithelial structure (inferred from the location and morphology of the DAPI-stained nuclei) and displayed continuous lumen formation (FIG. 5F arrows), while extensions projecting into the surrounding matrix were cord-like (FIG. 5F arrowheads).

### Effect of FGF10/7/2 on Construct Viability and Gene Expression

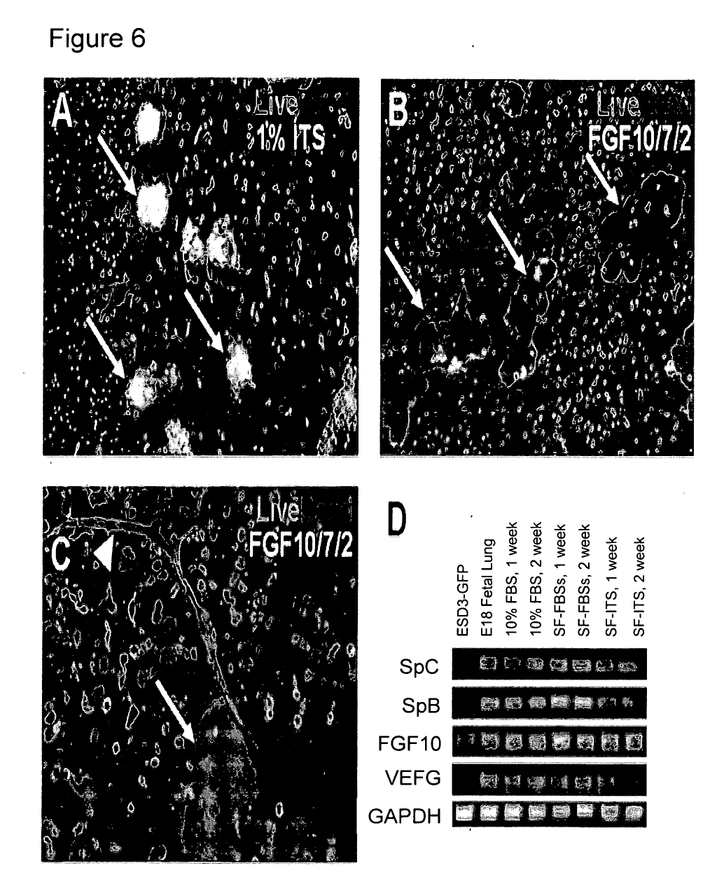

Results suggested that growth/viability and differentiation of epithelial cells present in FPC cultured in collagen gels in basal media supplemented with either 10% FBS or 1% ITS were inferior to parallel cultures in Matrigel (unpublished observations). The following experiments were designed to determine whether supplementation of 1% ITS media with FGF10/7/2 would enhance epithelial viability. As depicted in FIG. 6, the constructs cultured in FGF10/7/2 for 7 days contained only sparse individual dead cells and exhibited high viability in the budding epithelial structures (FIG. 6B, arrows). Conversely, the large spherical aggregates found in 1% ITS alone contained significant numbers of dead cells (FIG. 6A, arrows). Visual comparison of these images suggests that the numbers of cells in the interstitial spaces between epithelial structures is increased in FGF10/7/2 cultures, which is consistent with increased counts of tropoelastin-positive cell numbers (FIG. 2M). Inspection at higher magnification confirmed a high degree of viability amongst the cells comprising the budding structures (FIG. 6C arrow) and elongating tubular structures (FIG. 6C, arrowhead).

To test whether FGF10/7/2 enhanced the expression of some of the genes involved in morphogenesis and lung epithelial differentiation, RT-PCR using total RNA isolated from cells cultured for 7 or 14 days was performed. As seen in FIG. 6D, expression of distal epithelial marker genes SpC and SpB, the mesenchymal-derived morphogen FGF10 and vascular endothelial growth factor A (VEGF) was detected in all constructs irrespective of the media and culture time, albeit at different levels relative to GAPDH.

### Expression of FGFR1 and FGFR2 in FPC Input Material

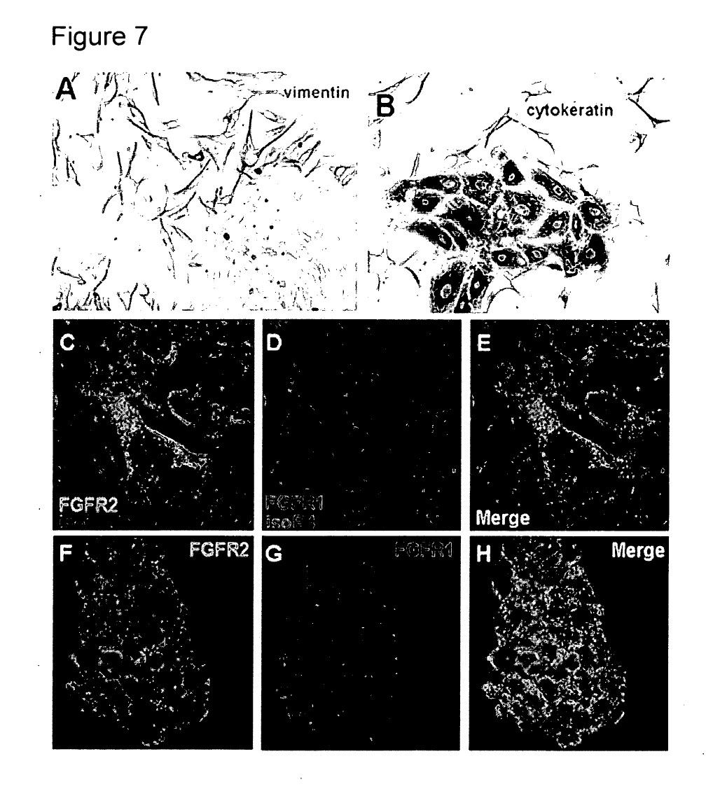

The following experiments were designed to examine whether the action of exogenous FGFs on FPC in the contructs is mediated via specific FGF receptors (FGFRs). In order to determine the presence of FGFRs on FPCs in the starting material at the time of FGF addition, FPCs were cultured overnight in 10% FBS and stained for FGFR1 and FGFR2. It has been demonstrated that these briefly cultured FPC contain islands of cytokeratin-positive epithelial cells which display tight cell-cell contact (FIG. 7B) surrounded by a more diffuse population of flattened mesenchyme expressing vimentin filaments (FIG. 7A), and that a sizeable fraction of these mesenchymal cells are isolectinB4+ ECs. Virtually all cells in the cultures, epithelial and mesenchymal, ubiquitously expressed both FGFR1 and FGFR2 protein (FIGS. 7C-7H).

### Tissue Engineered Model of Fetal Distal Lung Tissue

Tissue engineering aims at the development of tissue constructs for therapeutic purposes, as an alternative to organ transplantation. The results presented herein demonstrate that lung tissue engineering provides the field of lung biology with high fidelity 3-D tissue models. The establishment of organotypic fetal lung cell culture models has been reported (Douglas, et al., 1976, In Vitro 12: 373-381; Douglas, et al., 1976, Am Rev of Resp Disease 113: 17-23; Nakamura, et al., 2000, Am J Physiol Lung Cell Mol Physiol. 278(5): L974-80; Paszek, et al., 2005, Cancer Cell. 8(3): 241-54; Schwarz, et al., 2004, Am J Respir Cell Mol Biol. 30(6): 784-92), however, these models do not offer a 3-D organotypic cell culture model containing epithelial, endothelial and mesenchymal cells. The results presented herein demonstrate that differential effects of FGF10, FGF7 and FGF2 on histiotypic distal lung morphogenesis in 3-D collagen gel constructs in vitro.

None of the earlier works embedded cells in 3-D gels which allows for the establishment of true 3-D cell polarity. Furthermore, at the collagen concentrations used, the system described herein provides a mechanical environment more similar to that of soft tissues than rigid tissue culture plastic (Paszek, et al., 2005, Cancer Cell. 8(3): 241-54). The importance of compliant 3-D culture in hydrogels and the drawbacks of 2-D culture on plastic, for modeling of tissues with an epithelial component have been previously described (Lee, et al., 2007. Nat Methods 4(4): 359-65).

In the 3-D constructs presented herein, concerted epithelial and endothelial morphogenesis was impacted by organotypic coculture and addition of exogenous FGFs. However, it has been demonstrated that coculture and serum-free culture with FGF10/7/2 alone was insufficient to induce epithelial morphogenesis or maintain SpC gene expression in extended cultures on synthetic polymer scaffolds. Furthermore, in the 3-D collagen gel constructs, endogenous signaling elaborated in serum-free culture in the absence of exogenous FGFs was insufficient to induce epithelial or endothelial morphogenesis (FIG. 5A). An increase in the number of dead cells in cultures maintained with 1% ITS only (FIG. 6A) suggests that in the absence of serum, exogenous FGF10/7/2 function in part as survival/mitogenic factors for FPC cultured in collagen gels. The data presented herein suggest that FGF10/7 alone induce a ˜4 fold increase in epithelial cell numbers in AFUs, while FGF2 alone induces a similar ˜4 fold increase in mesenchymal cell numbers, which is combined additively in FGF10/7/2 cultures (FIG. 2M). The data presented herein suggest that FGF10/7/2 enhanced viability and proliferation of FPC in 3-D culture, while the mechanospatial cues present in compliant hydrogels, such as Matrigel and collagen gels, appear to allow for a morphogenic response. By contrast, the response of these cells to rigid polymer scaffolds is similar to 2-D culture. Therefore, a 3-D matrix, permissive to cell-cell and cell-growth factor interactions in an engineered system, provides an environment that satisfy basic biochemical and mechanical requirements.

Both endoderm-derived lung epithelium and the lung mesenchyme express FGFR1 and FGFR2; however epithelial cells express the “b isoforms”, while cells of mesenchymal origin express the “c isoforms”, and this confers ligand specificity (Ornitz, et al., 1996, J Biol Chem. 271(25): 15292-7; Shannon, et al., 2004, Annu Rev Physiol 66:625-45; White, et al., 2006, Development 133(8): 1507-17; Zhang, et al., 2006, Development 133(1):173-80). Studies using engineered cell lines that expressed individual FGFR isoforms revealed that FGF10 and FGF7 bind only FGFR2b, while FGF2 binds FGFR1b, FGFR1c, and FGFR2c (Ornitz, et al., 1996, J Biol Chem. 271(25): 15292-7). The results presented herein demonstrate that virtually all of the FPC tested express FGFR1 and FGFR2 protein (FIG. 7). Without wishing to be bound by any particular theory, it is believed that exogenous FGF10 and FGF7 signal exclusively to epithelial cells through FGFR2b. Similarly, it is believed that FGF2 signals to both mesenchymal and epithelial isoforms of FGFR1 and FGFR2, with preference for mesenchymal isoforms (Ornitz, et al., 1996, J Biol Chem. 271(25): 15292-7). This notion is supported by the increase in mesenchymal proliferation (tropoelastin-positive cell numbers) and EC network assembly in response to FGF2, and the specific effect of FGF10/7 on epithelial proliferation (epithelial cells/AFU) and budding, which are combined upon co-supplementation with FGF10/7/2 (FIGS. 2 and 4).

In line with its definitive role in inducing epithelial branching in vivo (Min, et al., 1998, Genes Dev 12(20): 3156-61), FGF10 significantly enhanced bud formation in the in vitro model; an effect that was not further enhanced by co-supplementation with FGF7 and FGF2 (FIGS. 1 and 2). This supports findings showing that exogenous FGF10 induces generalized epithelial budding in mesenchyme-free cultures in vitro, and rescues alveolar growth in a nitrofen-induced model of pulmonary hypoplasia in rats (Schuger, et al., 1996, Dev Biol. 179(1): 264-73). In the described herein, FGF10 is supplemented homogenously in the medium; however there is also endogenous FGF10 gene expression by mesenchymal cells in the preparations (FIG. 6D). This could possibly lead to the elaboration of local gradients, although the budding response appears to be general and not patterned in any way relative to other cells in the constructs. Co-supplementation of FGF10/7 did not significantly increase measured AFU areas and budding compared to FGF10 cultures (FIG. 1G), however AFUs in FGF10/7 cultures appeared more dilated (FIG. 1E vs. 1B), consistent with a proposed role for FGF7 in epithelial dilation (White, et al., 2006, Development 133(8): 1507-17). The RT-PCR results (FIG. 6D) demonstrating expression of the epithelial differentiation marker genes, SpC and SpB, across conditions suggest that baseline epithelial cytodifferentiation is retained in 3-D organotypic cultures and may be enhanced, but is not induced by exogenous FGF supplementation. The results suggest that the enhanced proSpC immunoreactivity observed in FGF10, FGF10/7 and FGF10/7/2 cultures (FIG. 2) may reflect increased proliferation of SpC-expressing cells present in the input material or enhanced paracrine signaling in these conditions.

Developmental interactions between lung epithelial and endothelial cells (Hislop, 2002, J. Anat. 201: 325-34) have not been studied nearly as extensively as interactions between lung epithelium and lung mesenchyme. However, tissue recombination experiments have demonstrated that lung epithelium is required for differentiation of distal lung mesenchymal progenitors into endothelial cells, suggesting that the epithelium may orchestrate distal pulmonary vasculogenesis (Gebb, et al., 2000, Dev Dyn 217(2): 159-69). Endothelial cells are required for development of the liver (Matsumoto, et al., 2001, Science 294(5542): 559-63) and pancreas (Lammert, et al., 2003, Mech Dev 120(1): 59-64), even prior to establishment of perfused vasculature, suggesting an instructive role for the endothelial cell in organogenesis of these endoderm-derived tissues. Although such a distinct role has not yet been established in lung development, evidence illustrating the potential instructive role of vascular development in regulating lung epithelial development has been reported in both in vitro (Schwarz, et al., 2004, Am J Respir Cell Mol Biol. 30(6): 784-92) and in vivo systems (Zhao, et al., 2005, Mech Dev. 122(7-8): 877-86). For example, Schwarz et al. reported that ablation of endothelial network formation by endothelial-monocyte activating polypeptide (EMAP) II inhibited formation of quasi-3D cystic epithelial aggregates (Schwarz, et al., 2004, Am J Respir Cell Mol Biol. 30(6): 784-92). Importantly, this study highlighted that EMAP II enhances the expression of fibronectin but not laminin, which may indicate that EMAP II inhibits epithelial development via alteration in the balance of extracellular matrix molecules, rather than a vascular-derived signal. Similarly, in murine embryonic lung allografts transplanted into the renal capsule, inhibition of vascular development using soluble VEGFR1 resulted in decreased vascular and saccular epithelial development (Zhao, et al., 2005, Mech Dev. 122(7-8): 877-86). However, since lung epithelial cells express VEGFRs (Compernolle, et al., 2002, Nat Med. 8(7): 702-10) this effect may also have been a direct inhibitory effect on epithelial proliferation or differentiation, and does not unequivocally support the notion that discrete vascular signals regulate epithelial growth and branching. More recent experiments using sonic hedgehog (SHH) deficient embryonic lung explants demonstrated that stimulation of vascular development with exogenous angiogenic factors, angiopoietin-1 and FGF2, promoted increased epithelial branching morphogenesis (van Tuyl, et al., 2007, Dev Biol. 303(2):514-526). However, since these angiogenic factors also increased mesenchymal proliferation, it is not clear in the study by van Tuyl et al. (van Tuyl, et al., 2007, Dev Biol. 303(2):514-526) whether increased epithelial branching was caused by enhanced mesenchymal signaling or increased vascularization. In the system described herein, robust epithelial-endothelial interfacing was observed in FGF10/7/2 cultures; however it has been demonstrated that the individual FGFs have differential effects on epithelial and mesenchymal cells. Addition of any single FGF resulted in significant epithelial growth (FIGS. 1G and 2M) and EC elongation/interconnection, however robust epithelial budding and uniform EC network assembly were only observed in parallel in FGF10/7/2 cultures. No significant differences in epithelial cell numbers or buds per AFU were observed when comparing FGF10/7 and FGF10/7/2 cultures (FIGS. 1 and 2), despite increased vascular development (FIG. 4G) and mesenchymal proliferation (FIG. 2M) observed in FGF10/7/2 cultures. Importantly, FGF2 only cultures, in which enhanced mesenchymal proliferation (FIG. 2M) was accompanied by uniform endothelial network formation (FIG. 4F), did not display robust epithelial proliferation and budding, when compared to FGF10/7 cultures (FIGS. 1G and 2M). It is believed that FGF2-induced vascular development (tubular morphogenesis) in the system results from a combination of both direct effects on EC via FGFRs (FIG. 7) and indirect effects, e.g. via increased mesenchymal cell numbers (FIG. 2M), which in turn elaborate increased levels of angiogenic factors. It is known that pulmonary mesenchymal cell-derived VEGFs contribute to pulmonary vascular development (Greenberg, et al., 2002, Dev Dyn. 224(2): 144-53). Preliminary comparison of network complexity in FGF10/7/2 vs. FGF2 cultures, which indicated an apparent 2 fold increase in network complexity (data not shown), despite almost no differences in mesenchymal proliferation (FIG. 2M), suggests that enhanced epithelial growth and budding induced by FGF10/7 positively influence vascular development. Distal epithelial cells express high levels of VEGF in vivo (Akeson, et al., 2003, Dev Biol. 264(2): 443-55), therefore increased epithelial cell numbers in FGF10/7/2 cultures likely results in increased levels of proangiogenic paracrine signaling. This is further supported by the observation that in the presence of FGF10/7, which signal exclusively to epithelial cells, endothelial tubular network formation is initiated, which does not occur in baseline conditions. Therefore, it is believed that factors which promote epithelial and mesenchymal viability and proliferation, also increase paracrine signaling activity to EC, and positively impact vascular morphogenesis in this engineered system. The data presented herein suggest that mesenchymal-derived factors, such as FGF10/7, play a more significant role in promoting epithelial proliferation and budding than a potential vascularderived signal, and that increased epithelial growth and branching positively impacts vascular development.

In summary, organotypic fetal lung tissue constructs using E17.5 FPC and a combination of 3-D culture in collagen type I gels and a serum-free medium containing FGF10, FGF7 and FGF2. The combination of FGF10/7 mostly influenced epithelial budding and proliferation, while FGF2 alone promoted EC network assembly and induced mesenchymal proliferation. Importantly, EC network complexity increased upon co-supplementation with FGF10/7/2, suggesting positive contribution of increased epithelial budding and proliferation to vascular development. This in vitro model of fetal distal lung tissue is useful for investigating lung developmental biology, in particular dynamic epithelial-endothelial interactions and to dissect the role of mesenchymal cells in these processes. This model also lays the groundwork for development of tissue engineering-based therapies for lung augmentation in pediatric and potentially adult pulmonary medicine.

### Example 2

### In Vivo Pulmonary Tissue Engineering: Contribution

Intrapulmonary engraftment of engineered lung tissues provides a potential therapeutic approach for the treatment of pediatric and adult pulmonary diseases. The results presented herein demonstrate the successful in vivo generation of vascularized pulmonary tissue constructs. By way of example, the subcutaneous Matrigel plug model was used. Mixed populations of murine fetal pulmonary cells (FPCs) containing epithelial, mesenchymal, and endothelial cells (ECs) were isolated from the lungs of embryonic day 17.5 fetuses. FPCs were admixed to Matrigel and injected subcutaneously into the anterior abdominal wall of adult C57/BL6 mice to facilitate in vivo pulmonary tissue construct formation. Vascularization was enhanced by placing fibroblast growth factor 2 (FGF2)-loaded polyvinyl sponges into the hydrogel. After 1 week, routine histology and immunohistochemical staining for donor-derived epithelial cells and ECs as well as analysis of patent vasculature in the constructs following tail vein injection of fluorescein isothiocyanate-conjugated dextran were performed. In the Matrigel-only controls, some level of host infiltrate, but no measurable vascularization was detected. In the presence of FPCs, the constructs contained ductal epithelial structures and patent vasculature. In the absence of FPCs, exogenous FGF2 induced the formation of numerous patent blood vessels throughout the entire constructs. The combination of FGF2 with FPCs resulted in enhanced capillary density and abundant interfacing between developing epithelial and vascular structures. Significant findings of this study were that distal pulmonary epithelial differentiation (as assessed by the expression of prosurfactant protein C) can be maintained in vivo and that donor-derived ECs contribute to the formation of patent vessels that interface tightly with ductal epithelial structures.

The materials and methods employed in these experiments are now described.

### Materials and Methods

Isolation of FPCs

All animal procedures were carried out in accordance with a protocol approved by the Institutional Animal Care and Usage Committee (IACUC #16150). Timed-pregnant Swiss Webster mice were purchased from Charles River Laboratories (Wilmington, Mass.). Fetal lungs were harvested from pups at gestational day 17.5 as previously described (Mondrinos, et al., 2006, Tissue Eng, 12: 717-28; Mondrinos, et al., 2007, Am J Physiol Lung Cell Mol Physiol 293: L639-50). Briefly, isolated lungs were rinsed in 1×phosphate-buffered saline (PBS) (Cellgro, Herndon, Va.), minced, and digested with prewarmed 0.5% trypsin in 1×PBS for 20-25 min at 378C. Following the trypsin digestion, the enzymatic activity was quenched by addition of two volume equivalents of Dulbecco's modified Eagle's medium (DMEM) (Cellgro) containing 10% fetal bovine serum (FBS; Hyclone, Logan, Utah), followed by extensive trituration using a Pasteur pipette. The resultant homogenates were filtered through a nylon mesh (70 μm; BD Falcon, San Jose, Calif.) and centrifuged at 800 rpm for 5 min. The cell pellet was resuspended in 900 μL of distilled water for to lyse red blood cells, followed by addition of 100 μL 10× PBS. The cells were then pelleted again, resuspended in a defined volume of DMEM containing 10% FBS, and counted in a hemocytometer; viability was assessed by trypan blue exclusion. For cell tracking experiments, freshly isolated FPCs were loaded with 25 μM CMTPX CellTracker dye (Invitrogen, Carlsbad, Calif.) according to the manufacturer's protocol before admixing to liquid Matrigel (BD Biosciences, San Jose, Calif.).

Preparation of Matrigel Plugs and Surgical Implantation

Matrigel™ plugs were prepared in accordance with a protocol approved by the Institutional Animal Care and Use Committee (IACUC #02662), as described previously by Akhtar et al. (Akhtar, et al., 2002, Angiogenesis 5: 75-80). In brief, syngeneic C57/BL6 mice (Jackson Labs, Bar Harbor, Me.) were injected subcutaneously in the anterior abdominal wall with 500 μL of Matrigel (BD Biosciences) containing 5 million FPCs per milliliter at a volume ratio of one part cell suspension to nine parts Matrigel (MG+FPCs). Upon solidification of the Matrigel (˜5 min), an FGF2-soaked polyvinyl sponge preloaded with 100 ng FGF2 (Sigma, St. Louis, Mo.) was introduced (MG+FPCs+FGF2) into the construct via a small skin incision over the injection site and a second incision into the solidified constructs. Matrigel without cells (MG) and with FGF2-loaded polyvinyl sponges only (MG+FGF2) were prepared as controls. Animals were humanely killed, and the constructs were harvested at 7 days.

Characterization of Matrigel Plug Vascularization

Perfused vasculature on the surface of the constructs was visualized by tail vein injection of fluorescein isothiocyanate (FITC)-conjugated dextran [FITC-dextran, 2,000,000 MW, 5% (w/v), in PBS; Sigma] immediately before killing, as previously described (Akhtar, et al., 2002, Angiogenesis 5: 75-80). These high-molecular-weight “fixable” dextrans contain lysine residues that allow crosslinking and fixation by aldehydes. Five minutes after tail vein injection, the animals were humanely killed, and the implants were excised and fixed in 10% buffered formalin for 2 hours at room temperature and then overnight at 4° C. The FITC-dextran-labeled vasculature was viewed by low-power fluorescence microscopy of entire constructs. In addition, paraffin sections of the FITC-dextran-perfused constructs were prepared for quantification of vascular density within the constructs via the persistence of the fixable dextran within the lumina of patent blood vessels. Upon deparaffinization and rehydration of the sections, patent vessels were readily visible under the fluorescent microscope. In double-staining experiments using the CMTPX CellTracker dye, both engrafted donor FPCs and patent host vessels were readily visible in the sections. The total area of FITC-dextran-positive pixels was quantified using NIH ImageJ software.

Histology and Immunohistochemistry

Excised constructs were prepared for routine histology and immunohistochemistry in paraffin sections as previously described (Mondrinos, et al., 2006, Tissue Eng, 12: 717-28). General construct morphology was assessed by hematoxylin and eosin staining. Expression of specific proteins was probed by indirect immunohistochemistry. Primary antibodies used included cytokeratin (polyclonal rabbit; DAKO, Carpinteria, Calif.) to identify epithelial cells, and prosurfactant protein C (proSpC) (polyclonal rabbit; Chemicon, Temecula, Calif.) to label alveolar type II lineage epithelial cells. For antigen detection, either immunoperoxidase staining (using the DAKO AEC+HRP kit, red reaction product) according to manufacturer's protocol or fluorescent Alexa488-conjugated goat anti-rabbit secondary antibody (Invitrogen) was used. Fluorescent secondary antibodies were used for analyzing only those constructs that had not been perfused with FITC-dextran. Negative controls were processed identically, except that the primary antibodies were omitted. An endothelial-specific lectin, (Laitinen, et al., 1987, Histochem J 19: 225-34; Hyink, et al., 1996, Am J Physiol 270(5 Pt 2): F886-99) Alexa488-conjugated Griffonia simplicifolia isolectinB4 (isolectinB4; Invitrogen), was used for EC identification in select whole mount preparations. Paraffin processing disrupted the sugar-binding interactions of isolectinB4 therefore necessitating a whole mount approach to utilize this marker for EC identification. Briefly, after fixation, constructs were dissected into pieces approximately 5 mm (Golpon, et al., 2006. Curr Drug Targets 7: 737-41) in size, permeabilized with 0.25% Triton-X prepared in 1% bovine serum albumin containing 1× PBS for 2 hour at room temperature, and incubated overnight with a 10 μg/mL solution of Alexa488-isolectinB4. The stained samples were washed in three to four changes of 0.1% Triton-X containing 1×PBS over 4-6 hour, whole mounted on a microscope slide, and examined by laser scanning confocal microscopy.

Statistical Analysis

Quantitative image analysis using NIH ImageJ software was employed to measure relative levels of vascularization by measuring the area of FITC-dextran-positive pixels. For quantifying vascularization, fields of paraffin sections (200× magnification) from FITC-dextran-perfused constructs were binarized and the percentage of the total pixel area contributed by FITC-dextran signal was calculated. These measurements were performed from a minimum of 10 sections of constructs harvested from a minimum of 12 animals for Matrigel+FPCs and Matrigel+FPCs+FGF2, and 6 animals for Matrigel+FGF2. The statistical significance in individual comparisons between the aforementioned conditions was determined by Student's t-test, with p<0.05 being statistically significant.

The results of the experiments are now described.

### Histology of In Vivo-Engineered Pulmonary Tissue Constructs

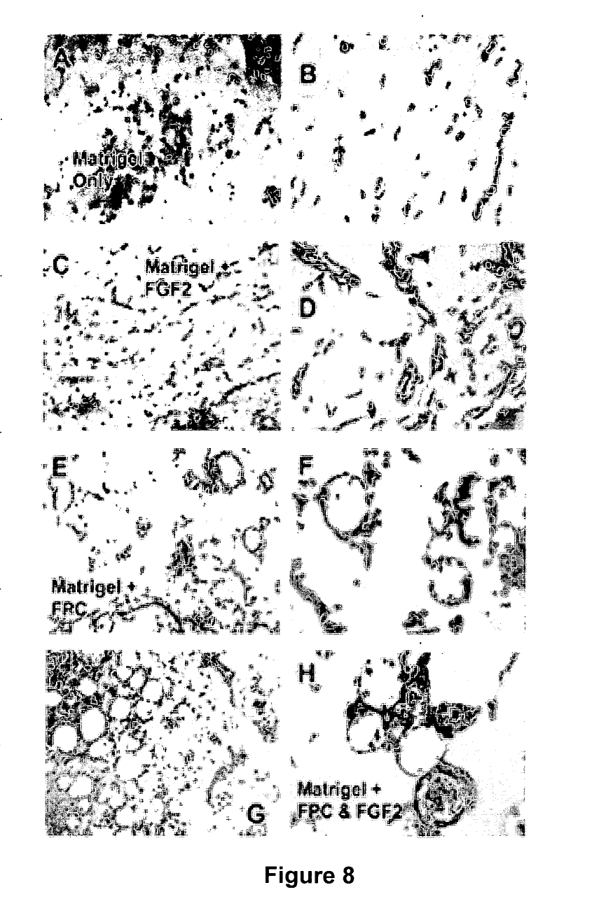

Histological analysis of control MG constructs without FPCs or FGF2 revealed considerable host infiltrate with little or no internal vascularization (FIGS. 8A, B). In the absence of FPCs (Matrigel+FGF2), incorporation of FGF2-loaded polyvinyl sponges led to abundant internal vascularization in addition to host infiltrates (FIGS. 8C, D). Incorporation of FPCs alone in the absence of FGF2 (Matrigel+FPCs) resulted in the formation of FPC-derived ductal structures, as well as the appearance of some internal blood vessels (FIGS. 8E, F). Constructs generated using FPC- and FGF2-loaded polyvinyl sponges (MG+FPCs+FGF2) demonstrated a significant increase in the number of patent blood vessels (FIG. 8G), which were often found juxtaposed with ductal epithelium (FIG. 8H).

### Immunohistochemical Analysis of Engrafted FPCs

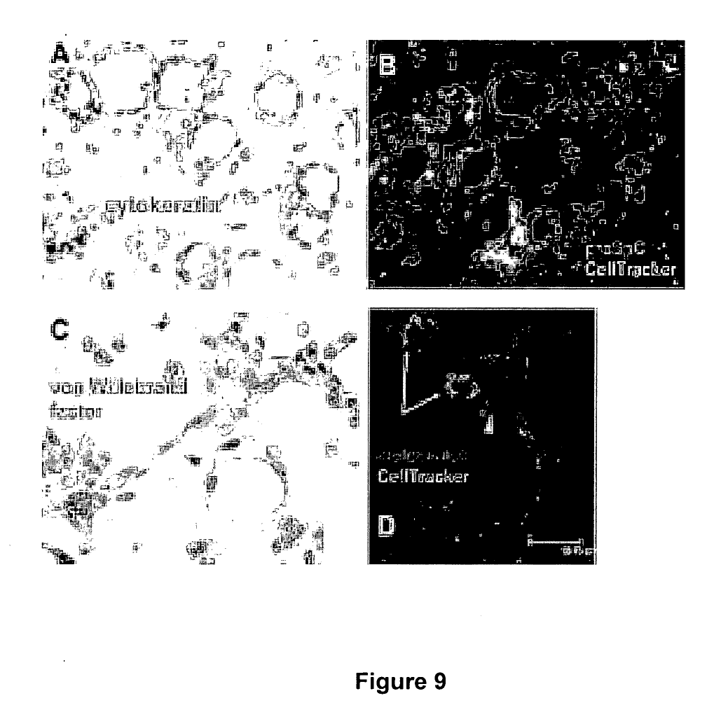

The epithelial nature of the cells lining ductal structures in hematoxylin and eosin-stained, FPC-containing constructs was confirmed by immunoperoxidase staining for the epithelial intermediate filament cytokeratin (FIG. 9A). The donor origin of the engrafted FPCs and their distal lung epithelial differentiation in the ductal structures was confirmed, respectively, by CellTracker labeling (orange) and fluorescent immunostaining for proSpC (green), the SpC gene product, which is expressed exclusively in cells of the type II alveolar epithelial lineage (FIG. 9B). As seen in FIG. 2B, most of the cells lining the ductal epithelium stain positive for proSpC (green/yellow). ECs present within the constructs were also identified by immunoperoxidase staining for von Willebrand factor, which highlights tubular endothelial structures amidst ductal epithelial structures (FIG. 9C). To assess the donor versus host origin of ECs, whole mounts of constructs containing CellTracker-labeled FPCs with the endothelial marker isolectinB4 were stained. The double-stained whole mounts were evaluated by laser scanning confocal microscopy. As seen in FIG. 9D, some of the cells lining vessel-like structures in the constructs were host derived (isolectinB4-positive and CellTracker-negative, arrowhead). However, other endothelial structures contain cells apparently of donor origin (CeilTracker-positive, FIG. 9D, arrows).

### Analysis of Patent Construct Vascularization

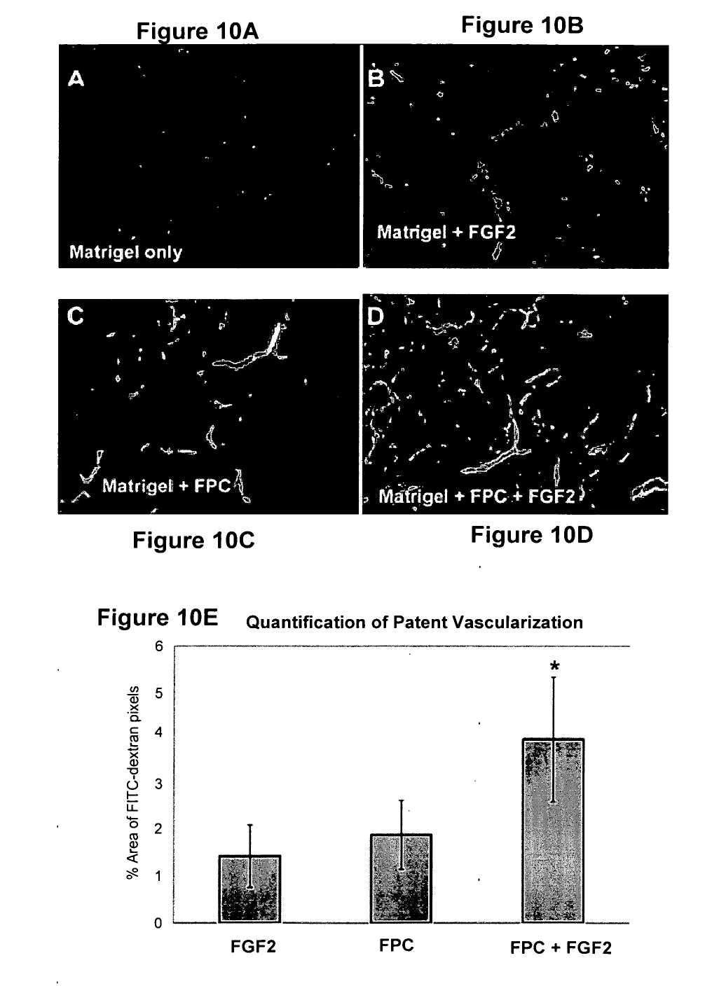

Fluorescent microscopy was used to assess the degree of vascularization in Matrigel plugs across experimental conditions. FITC-dextran tail vein injection allowed for visualization of patent, perfused vasculature both on the surface of freshly dissected constructs by gross fluorescent microscopy (not shown) and, subsequently, in transverse sections of paraffin-embedded samples (FIG. 10). Only sparse, small vessels were observed in the Matrigel-only controls (FIG. 10A). Matrigel+FGF2 constructs (FIG. 10B) and Matrigel+FPCs constructs (FIG. 10C) displayed similar levels of patent vessels, as seen qualitatively and confirmed by quantification of FITC-dextran pixel area (FIG. 10E). Matrigel+FPCs+FGF2 elicited an apparent additive effect, with significant increases in FITC-dextran pixel area (FIG. 10E), as well as a visually denser vascular network with more capillary-size vessels visible amidst larger diameter vessels (FIG. 10D).

### Donor FPC-Derived ECs Contribute to Patent Vascularization

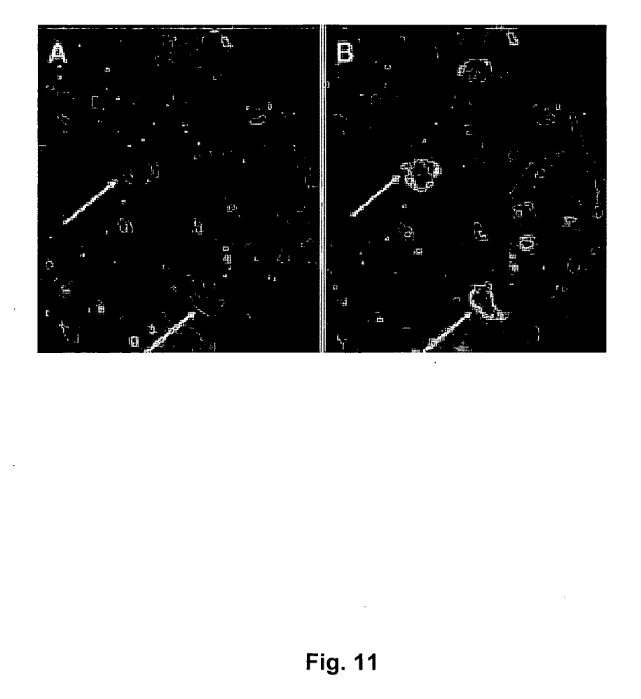

To evaluate whether donor-derived ECs present within the FPC mixture contribute to establishment of patent vasculature, constructs using FPCs prelabeled with CMTPX CellTracker dye were prepared to study the fate of donor-derived ECs. FIG. 11A (transverse section of a Matrigel+FPCs+FGF2 construct after FITC-dextran perfusion) shows CMTPX-labeled graft-derived cells, some of which form small lumen-containing structures, reminiscent of blood vessels (arrows). Merging with the FITC-dextran exposure of the same field reveals that the cells in these tubular structures are indeed ECs of FITC-dextran-perfused blood vessels, indicating that donor-derived ECs are part of a patent vasculature in the constructs that assembles into and/or anastomoses with the host circulation (FIG. 11B, arrows).

### Contribution of Donor-Derived Endothelial Cells to Construct Vascularization

Success in the budding field of distal lung tissue engineering requires the ability to generate a complex, 3D lung architecture and maintain lung epithelial differentiation in engineered systems. In addition to the importance of maintaining epithelial differentiation, in vivo vascularization of engineered pulmonary tissues upon implantation and connection to the host circulation is a prerequisite for graft survival and integration. In contrast to prior in vitro work in which purified alveolar epithelial cells were used to generate 3D cultures, (Adamson, et al., 1996, Am J Physiol 270(6 Pt 1): L1017-22; Sugihara, et al., 1993, Am J Pathol 142: 783-92; Bates, et al., 2002, Am J Physiol Lung Cell Mol Physiol 282: L267-76) the experiments described herein used mixed populations of fetal lung cells containing mesenchymal cells and ECs in addition to epithelium. A Matrigel plug assay was used as an exemplary system for investigating in vivo formation and vascularization of distal pulmonary tissue. Based on the data disclosed herein, it is believed that grafting of a mixed FPC population supports epithelial differentiation (SpC expression) and morphogenesis (formation of glandular structures) in an in vivo environment. It is believed that graft vascularization could be enhanced by exogenous FGF2, via increased host angiogenesis, and that graft ECs contribute to neovascularization in the constructs.

FPCs significantly enhanced neovascularization compared to Matrigel-only controls (FIGS. 8 and 10). Addition of FPCs alone promotes significant neovascularization, most likely as a result of angiogenic paracrine signals and/or contribution of donor-derived ECs. Previous studies indicated that distal vascular development and patterning in the lung is governed in part by vascular endothelial growth factor (VEGF) family ligands elaborated by epithelial (Akeson, et al., 2003, Dev Biol 264: 443-55) and mesenchymal cells, (Greenberg, et al., 2002, Dev Dyn 224: 144-53) both of which are present in our organotypic FPC mixture. In separate in vitro experiments, it has been determined that FPCs secrete physiological levels of VEGF-A (data not shown). Therefore it is likely that FPC-derived VEGF and other paracrine factors contribute to graft vascularization via influencing both donor-derived and host ECs. Incorporation of FGF2-soaked polyvinyl sponges in the absence of FPCs significantly enhanced vascularization relative to Matrigel only, to a similar degree as FPCs alone (FIG. 10). Interestingly, FPCs+FGF2 elicits an additive effect, with a significant two-fold increase relative to both FPCs and FGF2 alone (FIG. 10E). Since a syngeneic, not an immunodeficien, mouse model was used, a host inflammatory response to transplanted cells, Matrigel, and polyvinyl sponges likely contributes to enhanced vascularization. Indeed, there is a known correlation between inflammation due to the innate immune response and angiogenesis.(Naldini, et al., 2005, Curr Drug Targets Inflamm Allergy 4: 3-8; Frantz, et al., 2005, Circ Res 96: 15-26). In preliminary studies (not shown), it has been established that ˜20% of all cells present within FPC-containing plugs following 7 days in vivo were CD3+ lymphocytic infiltrate, a number that did not change in FPCs+FGF2 conditions, despite an approximately two-fold increase in patent vascularization. It is believed that the augmentation of neovascularization by exogenous FGF2-loaded sponges appears to be specific and does not result from increased inflammation.

Exogenous FGF2 significantly enhances construct vascularization above Matrigel-only controls (FIG. 10). FGF2 is a pleiotropic factor that elicits effects on lung epithelial cells, ECs, and mesenchymal cells via FGF receptors expressed by all these cell types. FGF2 has been reported to influence lung epithelial differentiation (Hyatt, et al., 2004, Am J Physiol Lung Cell Mol Physiol 287: L1116-26) and is also a potent angiogenic factor. (Sun, et al., 2004, World J Gastroenterol 10: 2524-8; Perets, et al., 2003, J Biomed Mater Res A 65: 489-97). In addition to its well-elucidated role in promoting sprouting angiogenesis both in vitro (Sun, et al., 2004, World J Gastroenterol 10: 2524-8) and in vivo, (Perets, et al., 2003, J Biomed Mater Res A 65: 489-97) FGF2 is also known to play a major role in vasculogenesis. Exogenous FGF2 induced in vitro hemangioblast differentiation of dissociated blastodisc cells that do not normally form blood islands, (Flamme, et al., 1992, Development 116: 435-9) and mediated vascular development in the embryonic chick chorioallantoic membrane. (Ribatti, et al., 1995, Dev Biol 170: 39-49). It has been demonstrated that exogenous FGF2 potently stimulates vascular plexus formation in 3D collagen gel cultures of FPCs in vitro. In addition, it has been demonstrated that exogenous FGF2 significantly enhances proliferation of mesenchymal cells present within the FPC mixture, which reciprocally enhances epithelial and endothelial development. Therefore, it is likely that exogenous FGF2 may manifest its effects in the system based on a combination of (i) stimulating sprouting of host vessels (angiogenesis), (ii) promoting by donorderived ECs the formation of a primitive vascular plexus (vasculogenesis) that anastomoses with the host vasculature, and (iii) enhancing mesenchymal and epithelial growth/proliferation, which positively impacts neovascularization via increased paracrine signaling.

There have been several successful attempts at engineering endothelial-lined microvessels in vitro and in vivo. (Nor, et al., 2001, Lab Invest 81: 453-63; Schechner, et al., 2000, Proc Natl Acad Sci USA 97: 9191-6; Wu, et al., 2004, Am J Physiol Heart Circ Physiol 287: H480-7; Koike, et al., 2004, Nature 428: 138-9) Recently, many groups have focused on engineering tissues containing differentiated, functional parenchyma and functional vascular structures in vitro and in vivo. (Kim, et al., 2005, J Korean Med Sci 20: 479-82; Yokoyama, et al., 2006, Am J Transplant 6: 50-9; Levenberg, et al., 2005, Nat Biotechnol 23: 879-84; Messina, et al., 2005, FASEB J 19: 1570-2; Birla, et al., 2005, Tissue Eng 11: 803-13; Brown, et al., 2006, Cell Transplant 15: 319-24) The generation of engineered vascularized bone, (Kim, et al., 2005, J Korean Med Sci 20: 479-82) hepatic, (Yokoyama, et al., 2006, Am J Transplant 6: 50-9) skeletal muscle, (Levenberg, et al., 2005, Nat Biotechnol 23: 879-84; Messina, et al., 2005, FASEB J 19: 1570-2) cardiac muscle, (Birla, et al., 2005, Tissue Eng 11: 803-13) and pancreatic tissues (Brown, et al., 2006, Cell Transplant 15: 319-24) by in vivo implantation of organ-specific parenchymal cells in the absence of grafted ECs has been previously reported. In all these cases, neovascularization is therefore likely mediated by angiogenesis from the host blood supply. For example, in experiments aimed at generating vascularized pancreatic islet tissue, Brown et al. (Brown, et al., 2006, Cell Transplant 15: 319-24) transplanted pancreatic beta cells in Matrigel within polycarbonate chambers that contained a surgically created AV loop, relying on host angiogenesis to develop the microvascular network of the graft.

There is increasing evidence that tissue construct vascularization may be enhanced by mixed vasculogenesis/ angiogenesis, provided that exogenously incorporated ECs can be coaxed to form vascular structures. (Nomi, et al., 2002, Mol Aspects Med 23: 463-83). A study by Levenberg et al. (Levenberg, et al., 2005, Nat Biotechnol 23: 879-84) reported that graft-derived endothelial structures present within in vitro-engineered skeletal muscle tissue constructs contribute to patent vessels in vivo. The enhanced in vitro vascularization and subsequent translation into function in vivo in the system described by Levenberg et al. (Levenberg, et al., 2005, Nat Biotechnol 23: 879-84) were attributed to their coculture conditions, in which ECs were coseeded with fibroblasts and skeletal muscle myoblasts. The in vivo model disclosed hereim employs a coculture approach, focusing on the role of heterotypic cell-cell interactions as a means of generating tissue constructs with an appropriately patterned vasculature, found in direct proximity to developing glandular epithelial structures (FIGS. 8H and 9C). This is significant to lung tissue engineering, where the developing circulation must interface with developing alveolar structures to establish the required architecture for efficient gas exchange. In addition to paracrine angiogenic activity resulting from coculture, contribution of FPC-derived ECs to neovascularization also accelerates establishment of patent vasculature throughout the 3D constructs in 7 days (FIG. 11). The FPCs contain approximately 15-20% ECs following brief 2D in vitro culture, (Mondrinos, et al., 2006, Tissue Eng, 12: 717-28) and it has been demonstrated that these ECs undergo vascular morphogenesis in vitro with exogenous FGF2. Without wishing to be bound by any particular theory, it is believed that optimization of combinatorial approaches employing addition of exogenous ECs, coculture with organ-specific parenchymal cells, and provision of exogenous proangiogenic factors to influence both donor-derived ECs and host angiogenesis are required to generate tissue constructs with robust, appropriately patterned vasculature.

The present results demonstrate the first report of formation of histiotypic alveolar-like structures in vivo, comprised of differentiated distal epithelial cells (proSpC expressing) forming ductal structures that are interfaced with a patent vascular network containing donor-derived ECs. The results presented herein demonstrate the ability to generate vascularized pulmonary tissue constructs in vivo utilizing Matrigel as a venue for transplantation of freshly isolated FPCs. Significantly, distal epithelial differentiation (proSpC expression) can be maintained in vivo in organotypic culture, and pulmonary ECs present in the organotypic mixture contribute to the formation of patent blood vessels. This model recapitulates the formation of structures reminiscent of alveolar forming units comprised of ductal epithelium tightly interfaced with the host circulation. Therefore, this model is useful for testing the effects of parameters such as exogenous growth factors, genetic modifications to engrafted cells, and addition of specific extracellular matrix molecules, as well as the utility of stem cell-derived populations of pulmonary cells in the process of distal lung tissue formation in vivo.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising a three dimensional scaffold and a population of fetal pulmonary cells (FPCs), wherein said composition is capable of supporting and maintaining the differentiation state of an alveolar epithelial cell.

2. The composition of claim 1, wherein said population of FPCs comprises epithelial, mesenchymal, and endothelial cells.

3. The composition of claim 1, wherein said cells are genetically modified.

4. The composition of claim 1, further comprising fibroblast growth factor (FGF), wherein said FGF is selected from the group consisting of FGF2, FGF7, FGF10, and any combination thereof.

5. The composition of claim 1, wherein said scaffold comprises a biocompatiable material selected from the group consisting of fibronectin, laminin, collagen, glycoprotein, thrombospondin, elastin, fibrillin, mucopolysaccharide, glycolipid, heparin sulfate, chondroitin sulfate, keratin sulfate, glycosaminoglycan, hyaluronic acid, proteoglycan, vitronectin, poly-D-lysine, polysaccharide, and any combination thereof.

6. An engineered three dimensional construct, wherein said construct is capable of supporting and maintaining the differentiation state of an alveolar epithelial cell.

7. The construct of claim 6, comprising a population of FPCs, wherein said population of FPCs comprises epithelial, mesenchymal, and endothelial cells.

8. The construct of claim 7, wherein said FPCs are genetically modified.

9. The construct of claim 6, comprising FGF, wherein said FGF is selected from the group consisting of FGF2, FGF7, FGF10, and any combination thereof.

10. The construct of claim 6, comprising cells that exhibit gene expression associated with induction of branching morphogenesis.

11. The construction of claim 10, wherein said gene is selected from the group consisting of surfactant protein C (SpC), SpB, FGF10, fibroblast growth factor receptor 2 (FGFr2), vascular endothelial growth factor A (VEGF), and any combination thereof.

12. The construct of claim 10, comprising a characteristic of a lung tissue, wherein said characteristic is selected from the group consisting of branching morphogenesis, distal lung epithelial cytodifferentiation, epithelial budding, epithelial growth, vascular development, and any combination thereof.

13. The construct of claim 6, wherein said construct is in a mammal.

14. The construct of claim 6, comprising a biocompatiable material selected from the group consisting of fibronectin, laminin, collagen, glycoprotein, thrombospondin, elastin, fibrillin, mucopolysaccharide, glycolipid, heparin sulfate, chondroitin sulfate, keratin sulfate, glycosaminoglycan, hyaluronic acid, proteoglycan, vitronectin, poly-D-lysine, polysaccharide, and any combination thereof.

15. A method of making an engineered three dimensional construct capable of supporting and maintaining the differentiation state of an alveolar epithelial cell, said method comprising seeding a scaffold with a population of FPCs to produce a seeded scaffold.

16. The method of claim 15, wherein said population of FPCs comprises epithelial, mesenchymal, and endothelial cells.

17. The method of claim 15, wherein said FPCs have been cultured in the presence of FGF for a period of time prior to seeding, wherein said FGF is selected from the group consisting of FGF2, FGF7, FGF10, and any combination thereof.

18. The method of claim 15, wherein said FPCs are seeded in the presence of FGF, wherein said FGF is selected from the group consisting of FGF2, FGF7, FGF10, and any combination thereof.

19. The method of claim 15, wherein said scaffold comprises a biocompatiable material selected from the group consisting of fibronectin, laminin, collagen, glycoprotein, thrombospondin, elastin, fibrillin, mucopolysaccharide, glycolipid, heparin sulfate, chondroitin sulfate, keratin sulfate, glycosaminoglycan, hyaluronic acid, proteoglycan, vitronectin, poly-D-lysine, polysaccharide, and any combination thereof.

20. An in vitro method for screening a test agent for the ability of said test agent to modulate the health of a lung tissue, said method comprising contacting said test agent to an engineered three dimensional lung tissue model and measuring the effect said test agent has on said model, wherein any alteration to the model is an indication that said test agent is able to modulate the health of a lung tissue.

21. The method of claim 20, wherein the test agent is selected from the group consisting of a chemical agent, a pharmaceutical, a peptide, a nucleic acid, and radiation.

22. The method of claim 20, wherein the test agent is a delivery vehicle for a therapeutic agent.

23. The method of claim 20 comprising determining the effect of the test agent on cell number, area, volume, shape, morphology, marker expression or chromosomal fragmentation.

24. The method of claim 20, further comprising the step of selecting an agent which has a desired effect on the lung tissue model.

25. A method of alleviating or treating a lung defect in a mammal, said method comprising administering to said mammal a therapeutically effective amount of a composition comprising a three dimensional construct capable of supporting and maintaining the differentiation state of an alveolar epithelial cell, thereby alleviating or treating said lung defect in said mammal.

26. The method of claim 25, wherein said construct comprises a population of FPCs, wherein said population of FPCs comprises epithelial, mesenchymal, and endothelial cells.

27. The method of claim 26, wherein said FPCs are genetically modified.

28. The method of claim 25, wherein said construct comprises FGF, wherein said FGF is selected from the group consisting of FGF2, FGF7, FGF10, and any combination thereof.

29. The method of claim 25, wherein said construct comprises cells that exhibit gene expression associated with induction of branching morphogenesis.

30. The method of claim 29, wherein said gene is selected from the group consisting of surfactant protein C (SpC), SpB, FGF10, FGFr2, vascular endothelial growth factor A (VEGF), and any combination thereof.

31. The method of claim 25, wherein said construct comprises a characteristic of a lung tissue, wherein said characteristic is selected from the group consisting of branching morphogenesis, distal lung epithelial cytodifferentiation, epithelial budding, epithelial growth, vascular development, and any combination thereof.
